(19) United States
(12) Patent Application Publication
(10) Pub. No.: US 2026/0344986 A1
(43) Pub. Date: Nov. 5, 2026

(54) HANDLE SYSTEMS AND ASSEMBLIES FOR MEDICAL DEVICES AND RELATED METHODS OF USE

(71) Applicant: Boston Scientific Scimed, Inc., Maple Grove, MN (US)

(72) Inventors: Joseph Phillip ALBINA, Hudson, MA (US); Collin MURRAY, Bolton, MA (US); Sean Powell, Holden, MA (US)

(73) Assignee: Boston Scientific Scimed, Inc., Maple Grove, MN (US)

(21) Appl. No.: 19/664,136

(22) Filed: Apr. 30, 2026

Related U.S. Application Data
(60) Provisional application No. 63/798,639, filed on May 2, 2025.
(60) Provisional application No. 63/882,625, filed on Sep. 16, 2025.

(51) Int. Cl. A61B 1/005; A61B 1/00

(52) U.S. Cl. CPC A61B 1/0052; A61B 1/0004; A61B 1/00045; A61B 1/0057

(57) ABSTRACT

A medical device includes a handle; a shaft extending distally from the handle and including an articulation portion and a distal tip; an actuator disposed on the handle and configured to control articulation of the articulation portion; a pulley disposed within the handle; a first and second wire; and a motor disposed within the handle and operatively connected to the pulley. A proximal end of each of the first wire and the second wire is coupled to the pulley. A distal end of each of the first wire and the second wire extends to the articulation portion. In response to manipulation of the actuator in a first actuation direction, the motor is configured to rotate the pulley, causing the proximal end of the first wire to move proximally and a proximal end of the second wire to move distally, such that the articulation portion articulates in a first articulation direction.

### CROSS-REFERENCE TO RELATED APPLICATIONS

This application claims the benefit of priority of U.S. Provisional Patent Application No. 63/798,639, filed on May 2, 2025, and of U.S. Provisional Patent Application No. 63/882,625, filed on Sep. 16, 2025, each of which is incorporated herein by reference in its entirety.

### TECHNICAL FIELD

The present disclosure relates to medical device handles, and more particularly to handles for a steerable medical device with electromechanical controls and, in some aspects, an integrated display.

### BACKGROUND

In a medical procedure, a user may insert a medical device, such as an endoscope or other type of scope, into a body lumen of a subject. The user may navigate the distal tip to a location within the lumen where the procedure is to be performed. Articulation and/or actuation of the distal tip may be controlled via one or more actuators on a handle of a traditional medical device. For example, levers, knobs, or the like may be used to actuate wires that are coupled to an articulation joint of the medical device. Furthermore, a lever or other actuator may be used to actuate an element of the distal tip, such as an elevator. A need exists for improved actuation mechanisms for medical device handles.

### SUMMARY

Each of the aspects disclosed herein may include one or more of the features described in connection with any of the other disclosed aspects. For example, aspects of this disclosure relate to, among other things, handle systems and assemblies for medical devices and methods of use. Aspects of this disclosure pertain to handles of medical devices, such as endoscopes or other scope devices, and related methods.

According to some aspects, a medical device may include a handle, a shaft extending distally from the handle and including an articulation portion and a distal tip, an actuator disposed on the handle and configured to control articulation of the articulation portion, a pulley disposed within the handle, a first wire and a second wire, and a motor. A proximal end of each of the first wire and the second wire is coupled to the pulley. A distal end of each of the first wire and the second wire extends to the articulation portion. The motor is disposed within the handle and operatively connected to the pulley. In response to manipulation of the actuator in a first actuation direction, the motor is configured to rotate the pulley, causing the proximal end of the first wire to move proximally and a proximal end of the second wire to move distally, such that the articulation portion articulates in a first articulation direction.

The handle device discussed above and elsewhere may include any of the following features alone or in any combination. The pulley is a first pulley and the motor is a first motor. The medical device further includes: a second pulley disposed within the handle, a second motor disposed within the handle and operatively connected to the second pulley, and a third wire and a fourth wire. A proximal end of each of the third wire and the fourth wire is coupled to the second pulley, and a distal end of each of the third wire and the fourth wire extends to the articulation portion. In response to manipulation of the actuator in a second actuation direction, different from the first actuation direction, the second motor rotates the second pulley, causing the third wire to translate proximally and the fourth wire to translate distally, thereby articulating the articulation portion in a second articulation direction, different from the first articulation direction. In response to manipulation of the actuator in a third actuation direction, each of the first pulley and the second pulley are rotated, thereby articulating the articulation portion in a third actuation direction, different from the first actuation direction or the second actuation direction.

The first pulley and the second pulley are adjacent to one another and have rotation axes that are parallel to one another but not coaxial. The pulley includes an axle extending from a first side of the handle to a second side of the handle, The motor is configured to rotate the pulley by rotating the axle. The actuator is a joystick. A degree of movement of the actuator corresponds to a degree of articulation of the articulation portion.

The actuator is a first actuator, and the motor is a pulley motor. The medical device further includes: an elevator disposed at the distal tip, a second actuator disposed on the handle, and an elevator motor. The second actuator is configured to control a position of the elevator. The elevator motor is configured to actuate the elevator in response to actuation of the second actuator. The medical device further includes an arm disposed within the handle, and an elevator pull wire having a distal end connected to the elevator and a proximal end directly or indirectly connected to the arm. The elevator motor is configured to rotate the arm to move the elevator pull wire proximally or distally to actuate the elevator. The second actuator is a slider or a lever.

The medical device further includes a third actuator. The third actuator is configured to adjust a speed of articulation of the articulation portion, a speed of actuation of the elevator, or both. The third actuator is a knob. An axis of rotation of the knob is approximately parallel to a longitudinal axis of the handle.

The medical device further includes a display integrated into the handle and configured to provide visual feedback regarding one or more of a position of the articulation portion, a position of the elevator, or a speed. The visual feedback regarding the position of the articulation portion includes a quadrant-based representation. The visual feedback includes a first symbol regarding the position of the articulation portion and a second symbol regarding a previous position of the articulation portion.

According to some aspects, a medical method includes receiving information related to a position of an articulation portion of a medical device; and displaying the position on of the articulation portion on a graphical user interface. The graphical user interface includes a quadrant illustration, a first symbol representing a current position of the articulation portion, and a second symbol representing a previous position of the articulation portion.

The medical method discussed above and elsewhere may include any of the following features alone or in any combination. The graphical user interface is displayed on a screen integrated into a handle of the medical device. The method further includes automatically moving the articulation portion to the previous position.

According to some aspects, a medical device includes: a handle; a shaft extending distally from the handle and including an articulation portion and a distal tip, wherein the distal tip includes an elevator; a first actuator disposed on the handle and configured to control articulation of the articulation portion; a second actuator disposed on the handle and configured to control a position of the elevator; and a motor configured to actuate the elevator in response to actuation of the second actuator.

The medical device discussed above and elsewhere may include the following feature. The medical device further includes a display integrated into the handle and configured to provide visual feedback regarding the position of the elevator.

The foregoing general description of the illustrative embodiments and the following detailed description thereof are merely exemplary aspects of the teachings of this disclosure and are not restrictive.

### BRIEF DESCRIPTION OF FIGURES

The accompanying drawings, which are incorporated in and constitute a part of this application, illustrate examples of this disclosure and together with the description, serve to explain the principles of the disclosure.

FIG. 1A illustrates a perspective view of an exemplary medical device, according to aspects of this disclosure.

FIG. 1B illustrates portions of a handle the medical device of FIG. 1A, revealing internal components of the handle, according to aspects of this disclosure.

FIG. 1C illustrates portions of the handle of the medical device of FIG. 1A, showing control and actuation elements, according to aspects of the present disclosure.

FIG. 1D illustrates an exemplary display configuration for use with the medical device of FIG. 1A, according to aspects of this disclosure.

FIG. 2 illustrates a perspective view of an alternative exemplary medical device handle, according to aspects of this disclosure.

FIG. 3 illustrates a perspective view of an alternative exemplary medical device, according to aspects of this disclosure.

FIGS. 4A and 4B illustrate schematic views of an internal motor assembly of the medical device of FIG. 3, according to aspects of this disclosure.

FIG. 5 illustrates a perspective view of a portion of the motor assembly of the medical device of FIGS. 3, 4A, and 4B, according to aspects of this disclosure.

FIG. 6 illustrates a schematic view of an alternative motor assembly of the medical device of FIG. 3, according to aspects of this disclosure.

FIG. 7 illustrates a perspective view of a further alternative motor assembly of the medical device of FIG. 3, according to aspects of this disclosure.

FIGS. 8-9B illustrate a system for controlling an accessory device using actuators of another medical device.

FIGS. 10A and 10B depict aspects of another medical device having a motor assembly.

### DETAILED DESCRIPTION

It may be understood that both the foregoing general description and the following detailed description are exemplary and explanatory only and are not restrictive of the invention, as claimed. As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from a user/toward a treatment site, and the term “proximal” refers to a direction toward a user. The term “approximately,” or like terms (e.g., “substantially”), includes values +/- 10% of a stated value. The term “or” includes “and/or.” Although endoscopes and duodensocopes are referenced herein, it will be appreciated that the medical devices described herein may include any type of scope, including, but not limited to, an endoscope, a ureteroscope, a duodenoscope, a cholangioscope, a gastroscope, an endoscopic ultrasonography (“EUS”) scope, a colonoscope, a bronchoscope, a laparoscope, an arthroscope, a cystoscope, or an aspiration scope. The medical devices described herein also include non-scope devices, such as a sheath, a catheter, a tome, or any type of accessory instrument having a shaft with a distal articulation portion and a handle.

Aspects of this disclosure provide handle systems and assemblies to facilitate treatment of a subject or improve performance of a distal portion of a medical device. The distal portion of the medical device, such as an endoscope or duodenoscope, may be inserted into a body lumen of a subject (e.g., into a duct or lumen of a patient) in order to perform a medical procedure (e.g., an endoscopic retrograde cholangiopancreatography procedure). The distal portion may include a distal tip and an articulation portion. The articulation portion may include an articulation joint and one or more articulation members configured to articulate or actuate the articulation portion in one or more directions (e.g., articulating, or moving, the distal tip left, right, up, or down). Such movements may be referred to herein as articulation or actuation directions.

The distal portion, including the distal tip, of the medical device may further include one or more assemblies, each including various electronic components, such as illumination or lighting elements (e.g., light emitting diodes (LEDs), optical fibers (e.g., plastic optical fibers (POFs), other light guides, or a combination thereof), imaging elements (e.g., cameras, other components having imagers, or other optical elements such as lenses), or other electronic components (e.g., capacitors, diodes, resistors, etc.). In some aspects, the distal tip may include an elevator. As will be described in further detail below, aspects of the distal portion, including the elevator, may be controlled via one or more actuators of the handle.

Ergonomic challenges in conventional handle designs may significantly impact the user experience during prolonged medical procedures. The discomfort and awkwardness in holding and manipulating these handles can lead to hand fatigue, muscle strain, and potential repetitive stress injuries for healthcare professionals who frequently use these devices.

In some aspects, it may be desirable for the handle of medical devices to include one or more electromechanical actuators configured to articulate or actuate various aspects of the distal portion. These actuators may reduce fatigue and ergonomic discomfort for a user. Furthermore, these actuators may enable very precise movements of the distal portion, which can be advantageous during medical procedures. For example, precise articulation and actuation may allow for more accurate navigation through complex anatomical structures, potentially reducing the risk of tissue damage or patient discomfort. Additionally, fine control over the distal portion may enhance the user's ability to perform delicate tasks, such as biopsy collection or targeted drug delivery. Furthermore, the ability to make minute adjustments to the position or orientation of the distal portion may facilitate access to hard-to-reach areas within the body, potentially expanding the range of procedures that can be performed using the medical device.

Furthermore, the disclosed handles may allow for user customization (e.g., of sensitivity of inputs) or periodic updates over a network connection. The disclosed handles may reduce components of a handle as compared to conventional devices, thereby improving manufacturability. The motorized actuators may integrate with artificial intelligence or computer vision models, such that software may automatically operate motors or other electronics, including satellite mounted motors or electronics or motors or electronics included in or on the medical device or medical system, to, for example, stabilize the distal tip of the medical device. In aspects, if a user jostles or wiggles the scope and the handle moves, the distal tip (including a camera) may not move because the software may automatically compensate for such unintentional movement and automatically actuate the articulation joint. In other examples, a software program may automatically drive sub-routines for complex procedures, such as endoscopic submucosal dissection (ESD). In examples, the disclosed systems and assemblies may have a feature that automatically moves the tip back and forth in any plane without requiring manipulation of two actuators simultaneously. The disclosed systems and assemblies may collect data regarding users’ navigation techniques and trends. For example, this data may be used for development of robotic systems or as training data for other systems.

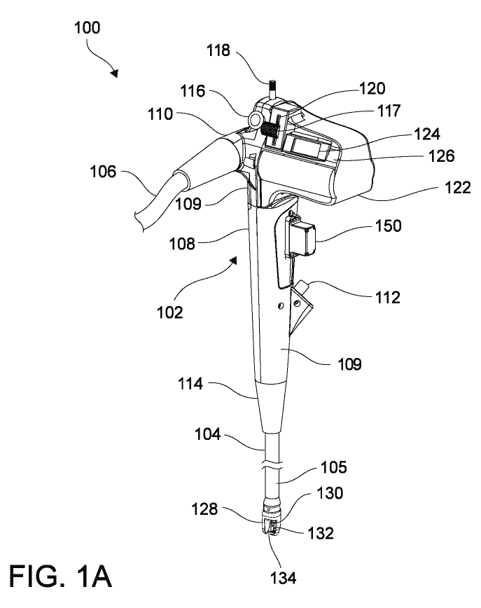

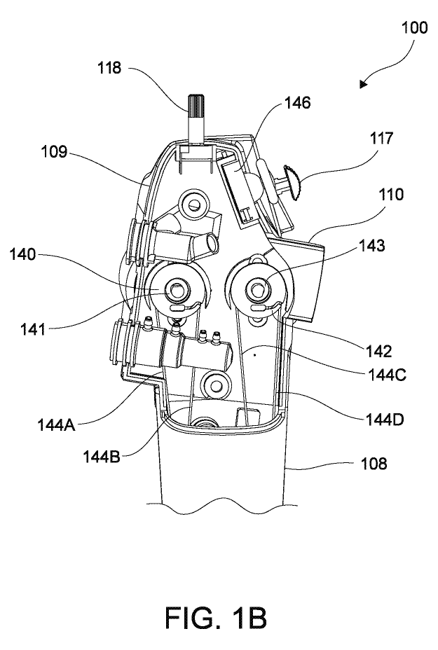

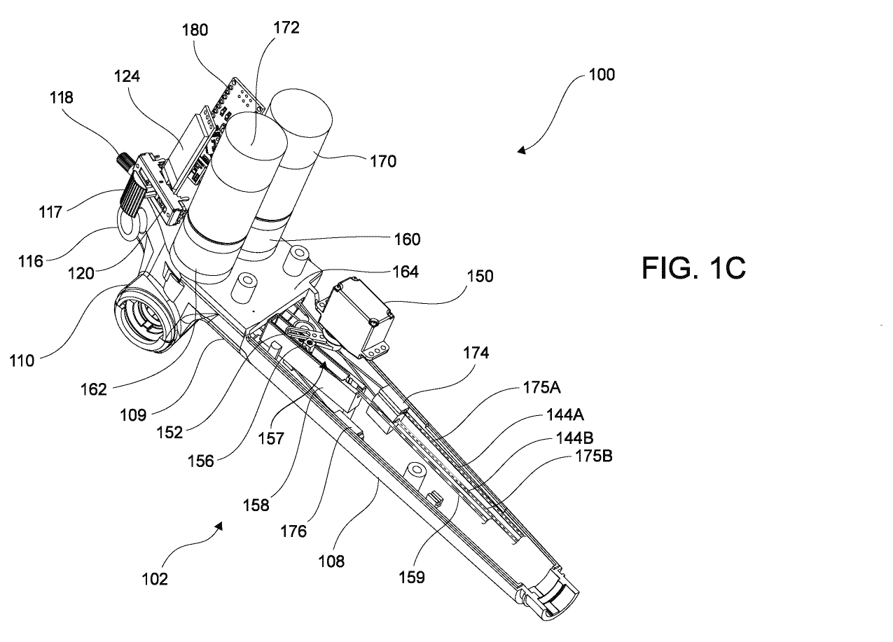

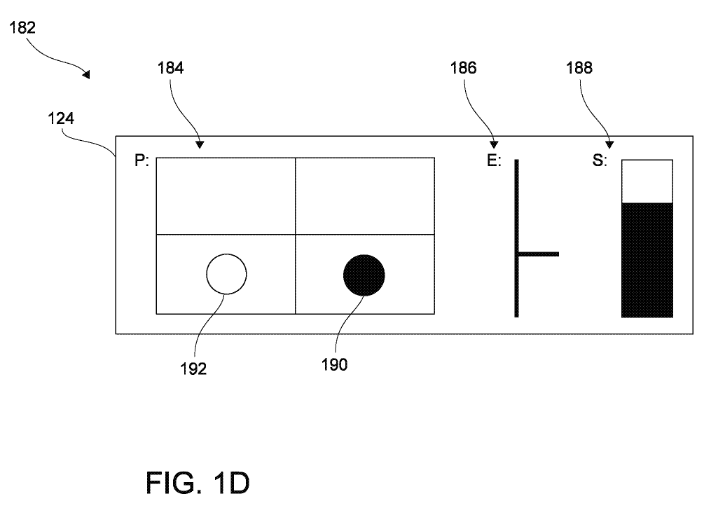

FIGS. 1A-1C illustrate various views of a medical device 100. For example, FIG. 1A illustrates a perspective view of medical device 100; FIG. 1B illustrates a view of a first side of a handle 102 of medical device 100 with a portion of a housing of handle 102 removed; and FIG. 1C illustrates a view of a second, opposite side of handle 102 of medical device 100 with a portion of the housing of handle 102 removed. Particularly when describing handle 102 and internal components of handle 102 of medical device 100, all of FIGS. 1A-1C may be referred to in the following description.

In addition to handle 102, medical device 100 may further include a shaft 104 extending distally from handle 102 and an umbilicus 106. Handle 102 may include a gripping portion 108 formed by one or more handle housing pieces 109. Handle housing pieces 109 may be formed from a rigid material, such as a medical-grade polymer, metal, or composite material, and may be configured to contain or provide structural support for the internal components of handle 102, such as the components shown in FIGS. 1B and 1C. Handle housing pieces 109 may include ergonomic contours (e.g., protrusions or indentations) and surface features (e.g., roughened surfaces, tacky surfaces, etc.) designed to enhance grip comfort and control during extended medical procedures. In some aspects, handle housing pieces 109 may be formed as two or more separate pieces, such as a left housing portion and a housing shell portion, that can be assembled to enclose the internal components of handle 102. In each of FIGS. 1B and 1C, one or more of handle housing pieces 109 have been removed to reveal internal components of handle 102, described in further detail below.

Handle 102 may include an umbilicus port 110 for connecting to umbilicus 106. Umbilicus 106 may provide connections for tubing, wiring, or cabling between handle 102 and one or more external fluid or electronic sources. For example, umbilicus 106 may be configured to connect handle 102 to fluidic sources such as fluidic pumps or suction sources. Additionally or alternatively, umbilicus 106 may connect handle 102 to one or more electronic sources including computers, controllers, or displays. Through umbilicus 106, fluids, data, or power may be transferred between handle 102 and these external sources, enabling various functionalities of medical device 100 during operation (e.g., insufflation, aspiration, fluid delivery, image capture, illumination, etc.).

Shaft 104 may extend distally from handle 102 and may be configured to be inserted into a body lumen of a subject. A strain relief 114 may be positioned at the junction of handle 102 and shaft 104. Strain relief 114 may be designed to reduce stress concentration and prevent kinking or damage to internal components at this transition point. At least a portion of shaft 104 may be flexible, semi-flexible, or rigid.

A distal end portion of shaft 104 may include an articulation portion 105 and a distal tip 128 that is fixed to a distal end of articulation portion 105. In some aspects, articulation portion 105 may include an articulation joint that is articulated or steered via controls (e.g., actuators) on handle 102, allowing a user to maneuver distal tip 128 within the body. For example, articulation portion 105 may be configured to bend in one or more directions relative to a longitudinal axis of shaft 104 (e.g., left, right, up, or down).

Distal tip 128 may include an imaging device 130 and an illumination device 132. Imaging device 130 may be capable of capturing still images or video footage. Such footage may be transmitted to handle 102 or an external display for viewing by the user (e.g., via umbilicus 106 or other means). Imaging device 130 may be side-facing (as shown) or forward-/distal-facing. Illumination device 132 may include one or more light sources, such as light-emitting diodes (LEDs) or optical fibers. Illumination device 132 may provide light to illuminate an area around distal tip 128, thereby enhancing visibility for the imaging device 130 and the user.

Distal tip 128 may further include an elevator 134 configured to change the angle or direction of instruments or tools that are passed through medical device 100. For example, an instrument or tool may be inserted through a port 112 of handle 102 that is in communication with a lumen or a working channel of shaft 104. The instrument or tool may be extended through the lumen or working channel and out of a distal opening of distal tip 128. In some aspects, elevator 134 may be configured to enable the user to adjust the position or orientation of instruments exiting the distal tip 128 during a procedure. Described in further detail below, a position of elevator 134 or a speed at which elevator 134 may be actuated may be controlled by one or more actuators on handle 102.

Handle 102 may include one or more actuators for controlling various functions of the medical device 100. Such actuators may include a first actuator 116, a second actuator 117, or a third actuator 118. Although three actuators are shown, medical device 100 may include fewer actuators or additional actuators. Such actuators may be buttons, knobs, sliders, levers, joysticks, switches, etc. Each actuator may be configured to control one or more functions of medical device 100. While specific functions for each actuator are described below, it should be understood that the actuators may be interchangeable or multifunctional. For example, first actuator 116 may be configured to accomplish the same functions as second actuator 117 or third actuator 118. Similarly, second actuator 117 may be configured to perform the same functions as first actuator 116 or third actuator 118. Third actuator 118 may likewise be configured to perform the same functions as first actuator 116 or second actuator 117.

In some aspects, first actuator 116 may be a joystick configured to control a position or configuration of articulation portion 105, and, therefore, a position of distal tip 128. For example, first actuator 116 may be configured to articulate, or bend, articulation portion 105 in one or more directions (e.g., up, down, left, or right relative to a longitudinal axis of shaft 104). In other aspects, first actuator 116 may be one or more rocker switches or knobs.

First actuator 116 may be electronically coupled to a first circuit board 146 (FIG. 1B). Circuit board 146 may be electronically connected to one or more motors 170, 172 (FIG. 1C) to control the operation of one or more pulleys 140, 142 within handle 102. For example, when a user manipulates first actuator 116, the movement is detected by circuit board 146, which then activates a first motor 170 to rotate a first pulley 140 or a second motor 172 to rotate a second pulley 142. Motors 170, 172 may include direct drive motors, satellite mounted motors, linear actuators, or any other type of motor configured to rotate pulleys 140, 142.

Moving first actuator 116 in one or more directions may result in one or both of first pulley 140 and second pulley 142 being rotated. For example, moving first actuator 116 in a proximal direction (e.g., toward a proximal end of medical device 100) may cause circuit board 146 to activate first motor 170 to rotate first pulley 140 clockwise, while moving first actuator 116 in a distal direction (e.g., toward a distal end of medical device 100) may cause first motor 170 to rotate first pulley 140 counterclockwise, or vice versa. Similarly, moving first actuator 116 to the left may cause second motor 172 to rotate second pulley 142 clockwise, while moving first actuator 116 to the right may cause second motor 172 to rotate second pulley 142 counterclockwise, or vice versa. Diagonal movements of first actuator 116—such as proximally and to the left, proximally and to the right, distally and to the left, or distally and to the right—may cause simultaneous rotation of both first pulley 140 and second pulley 142, allowing for combined movements of distal tip 128.

This system may also allow for proportional control, where the degree of movement of the first actuator 116 corresponds to the degree of articulation of articulation portion 105. For example, a small movement of first actuator 116 may result in a subtle articulation of articulation portion 105, while a larger movement of first actuator 116 may cause a more pronounced articulation. This feature may allow for both fine adjustments and larger movements as needed during a procedure.

Furthermore, this directional and proportional control of first actuator 116 and the articulation of articulation portion 105 may provide an intuitive control mechanism for the user. The user may be able to accomplish more precise and efficient navigation of articulation portion within the body using first actuator 116. In some cases, this intuitive control scheme may reduce the learning curve for users and may enable more accurate manipulation of the medical device 100 during procedures.

Each pulley 140, 142 may be connected to one or more pull wires 144A-D. One or more pull wires 144A-D may be individual pull wires or a single wire looped around a respective pulley 140, 142. Accordingly, rotation of each pulley 140, 142 may result in the translation of pull wires 144A-D proximally or distally. For example, proximal movement of a first pull wire 144A coupled to first pulley 140 may result in distal movement of a second pull wire 144B connected to the same first pulley 140. Pull wires 144A-D may extend distally from each pulley 140, 142 through handle 102 and shaft 104, towards distal tip 128. In some aspects, each pull wire 144A may be fixed to a distal end of articulation portion 105 or to a proximal end of distal tip 128. Translation of the pull wires 144A-D may result in the articulation of articulation portion 105 of shaft 104, and thus, movement of the distal tip 128. The term “pull wire” is used herein, but may be used to refer to a wire, a cable, a bundle of wires, a strand, or a filament.

Handle housing pieces 109 may further include a first block 174 (e.g., a first spring block) and a second block 176 (e.g., a second spring block) configured to maintain a relative position of pull wires 144A-D within handle 102, while still maintaining the ability for each pull wire to translate proximally or distally. In some aspects, a distal face of each block 174, 176 may provide a proximal stop for a respective coil pipe or other tube or sheath that each of pull wires 144A-D extends through. Only two coil pipes 175A, 175B are shown in FIG. 1C due to the orientation of medical device 100. Each block 174, 176 may be on an opposite side of handle 102 relative to one another and, in some aspects, may be integrally formed with handle. Two blocks 174, 176, rather than a combined block, may be used due to pulleys 140, 142 being positioned alongside one another, rather than coaxially.

Pulleys 140, 142 may be arranged adjacent to one another to allow enough room within the handle 102 for each pulley to have a respective motor (e.g., a respective pulley motor). Pulleys 140, 142 may have parallel but not coaxial axes of rotation. Respective motors 170, 172 for each pulley 140, 142 may be oriented coaxially with a rotational axis of each respective pulley 140, 142. Motors 170, 172 may be at least partially enclosed within a respective motor housing 160, 162. This configuration may help to protect motors 170, 172 from debris or fluids that may leak within handle 102.

Each pulley 140, 142 may include an axle 141, 143 rotationally fixed respectively thereto. Each axle 141, 143 may extend from a respective pulley 140, 142 on a first side of handle 102, through a dividing plate 164, and into a motor housing 160, 162 of a respective motor 170, 172. Axles 141, 143 may be coupled, directly or indirectly, to respective motors 170, 172. In some aspects, axles 141, 143 may be coupled to respective motors 170, 172 via worm gears, bevel gears, or the like. Accordingly, activation of motors 170 or 172 may result in the rotation of a respective axle 141 or 143, and, therefore, the rotation of a respective pulley 140 or 142. Dividing plate 164 may be integrally formed with handle housing pieces 109 or may be a separate component configured to separate at least a portion of handle 102.

Second actuator 117 may include a rocker switch, a slide potentiometer, a lever, a rotary potentiometer, or buttons configured to control a position of elevator 134 at distal tip 128. For example, second actuator 117 may adjust a position of elevator 134 up, down, or to any position in between. In some aspects, second actuator 117 may be a slider or lever configured to slide or pivot proximally or distally within a slot 120 of handle 102. For example, a proximal-most position of second actuator 117 within slot 120 may correlate to a fully closed/raised position of elevator 134, and a distal-most position of second actuator 117 may correlate to a fully open/lowered position of elevator 134, or vice versa. Any position of second actuator 117 between the fully proximal and fully distal positions may correlate to a partially open/raised or a partially closed/lowered position of elevator 134.

Second actuator 117 may be directly or indirectly connected to a third motor 150, such as a servo motor, a DC motor, or a linear actuator. Third motor 150 (also referred to herein as an elevator motor) may include a circuit board configured to translate a relative position of second actuator 117 to a position of elevator 134 or a speed at which second actuator 117 is moved to a relative speed at which elevator is moved. Accordingly, second actuator 117 may have directional and proportional control of elevator 134. For example, the degree of movement of the second actuator 117 may correspond to the degree of actuation of elevator 134. For instance, a small movement of the second actuator 117 may result in a subtle actuation of elevator, while a larger movement of the second actuator 117 may cause a more pronounced actuation. This feature may allow for both fine adjustments and larger movements as needed during a procedure.

This directional and proportional control of second actuator 117 and the actuation of elevator 134 may provide an intuitive control mechanism for the user. The user may be able to accomplish more precise movement of elevator 134. In some cases, this intuitive control scheme may reduce the learning curve for users and may enable more accurate manipulation of the medical device 100 during procedures.

Third motor 150 may be configured to actuate an arm 152 (e.g., a lever or a linkage). Arm 152 may be connected to a coupler 156. Coupler 156 may be movably disposed within a slot 157 of a track 158. Track 158 may be integrally formed with handle housing pieces 109 or may be a separate component installed within handle 102. Accordingly, when second actuator 117 is translated proximally or distally, that actuation is transmitted to third motor 150, which is configured to rotate arm 152 proximally or distally, accordingly. Rotation of arm 152 results in the proximal or distal translation of coupler 156 within slot 157 of track 158.

A proximal end of a pull wire 159 (also referred to herein as an elevator pull wire) may be coupled to coupler 156. Pull wire 159 may extend distally from coupler 156, through first a channel of first block 174. In some aspects, pull wire 159 may extend through a coil pipe, similar to coil pipes 175A, 175B. Pull wire 159 may exit through a distal end of handle 102 and extend through a lumen of shaft 104. A distal end of pull wire 159 may be coupled directly or indirectly to elevator 134. In such a way, translation of pull wire 159 proximally or distally, may result in elevator 134 being actuated.

Third actuator 118 may be a knob, dial, wheel, a rotary potentiometer, another rotational actuator, a lever, a slider, etc. An axis of rotation of third actuator 118 may be approximately parallel to a longitudinal axis of handle 102. In aspects, third actuator 118 may function as a speed or sensitivity control for the movements or actions initiated by first actuator 116 or second actuator 117. For example, when first actuator 116 is used to control the articulation of distal tip 128, the rotation of third actuator 118 may adjust the rate at which the articulation occurs or the sensitivity with which inputs from actuator 118 are transmitted. Additionally or alternatively, when second actuator 117 is used to control a position of elevator 134, rotation of third actuator 118 may adjust the rate at which the actuation occurs or the sensitivity of actuator 117. A clockwise rotation of third actuator 118 may increase the speed of articulation/actuation, while a counterclockwise rotation may decrease it, or vice versa. This speed or sensitivity control may allow for both rapid positioning and fine, precise adjustments of the distal tip 128 or elevator 134 as needed during a procedure.

In some aspects, the functionality of third actuator 118 may be context-sensitive, automatically adjusting its control parameters based on which other actuators are currently in use or based on the current position of medical device 100 within the subject. For example, quick movements of articulation portion 105 or elevator 134 may be inhibited when distal tip 128 is in a smaller body lumen of the subject. In such a way, perforations of distal tip 128 through a wall of the lumen or damage to the tissue surrounding distal tip 128 may be minimized.

In some aspects, third actuator 118 may offer discrete speed settings (e.g., low, medium, high) or may provide continuous variable speed control within a defined range. The actuator may include tactile feedback mechanisms, such as detents or variable resistance, to help the user gauge the current speed setting without needing to look away from the procedure.

In other aspects, third actuator 118 may be programmed to control multiple speed-related parameters simultaneously. For instance, third actuator 118 may be configured to adjust both the articulation speed of distal tip 128 and the movement speed of elevator 134 proportionally, maintaining a consistent feel across different functions of the device.

In some aspects, the automatic adjustment of third actuator 118 may be accomplished via one or more sensors or a force feedback loop. For example, during use, articulation portion 105 may be articulated and hit, or abut against, the surrounding tissue. When articulation portion 105 does hit the tissue, a resulting force may be detected by the system (e.g., via one or more sensors or via a force feedback loop) and parameters of the articulation of articulation portion 105 may be automatically adjusted.

The force feedback loop may incorporate one or more force sensors positioned along the articulation portion 105 or distal tip 128, including elevator 134. These sensors may be strain gauges, piezoelectric sensors, or other force-sensitive elements capable of detecting and measuring the forces exerted on the articulation portion 105 or distal tip 128 as it interacts with surrounding tissue. The sensors may continuously monitor the forces experienced by the articulation portion 105 and distal tip 128 during movement and articulation. When the articulation portion 105 encounters resistance from tissue, the force sensors may detect an increase in applied force. This force data may be transmitted back to the handle 102, where it may be processed by circuit board 146 or another dedicated processor. The processor may analyze the force data in real-time, comparing it to predetermined thresholds or using algorithms to interpret the nature and magnitude of the tissue interaction.

Based on this analysis, the system may automatically adjust various parameters of the articulation. For instance, if the detected force exceeds a certain threshold, the system may reduce the speed of articulation, increase the torque applied by motors 170, 172, or alter the articulation angle to navigate around the obstruction. These adjustments may be made by modifying the control signals sent to motors 170, 172, effectively changing the behavior of the articulation portion 105 without direct input from the user.

Similarly, when elevator 134 encounters resistance from an instrument or tool, force sensors may detect an increase in applied force. This force data may be transmitted back to the handle 102, where it may be processed. A speed or actuation force of elevator 134 may be automatically adjusted accordingly.

The force feedback loop may also provide haptic feedback to the user through first actuator 116 or second actuator 117. For example, when significant resistance is encountered, the system may increase the resistance felt by the user when manipulating the actuators, providing a tactile indication of the forces being experienced at the distal end of the device. The tactile indication may include a vibration, indicating to the user that a force threshold was exceeded, such as when distal tip 128 interacts with the surrounding tissue. In other aspects, the feedback may be auditory or visual. For example, a sound may be played or a notification may be displayed on an external display (e.g., a monitor) or an integrated display or user interface (e.g., a display 124), providing an indication to the user as to how much force was or is being applied to the tissue or instrument.

As shown in FIG. 1A, handle 102 may include an extension 122 extending laterally outward relative to a longitudinal axis of handle 102 at a proximal end of handle 102. Extension 122 may include a plurality of surfaces, including a surface 126, providing an area for user interaction or an information display, such as a display 124. In some implementations, surface 126 may be slightly recessed or raised relative to the surrounding areas of extension 122, potentially enhancing visibility or tactile differentiation for the user.

Display 124 may be incorporated on surface 126 of extension 122. Display 124 may comprise various types of screen technologies, such as LCD, OLED, or e-ink. The positioning of display 124 on extension 122 may allow for easy viewing by the user during use of medical device 100. This arrangement may enable the user to monitor important information or adjust settings without significantly altering their grip on handle 102 or shifting their focus away from the procedure.

In some implementations, display 124 may present various types of information relevant to the operation of medical device 100. This may include, but is not limited to, device status indicators, articulation angles of distal tip 128, elevator 134 position, imaging data from imaging device 130, or other procedural information. The content shown on display 124 may be configurable or context-sensitive, adapting to the current state or position medical device 100.

For example, referring to FIG. 1D, an exemplary display configuration 182 (e.g., a user interface) for the medical device 100 is illustrated. Display configuration 182 may be presented on display 124 of handle 102 of the medical device 100 or on an external display. In aspects, display configuration 182 may be displayed on an external display to which medical device 100 is coupled in a picture-in-picture arrangement. For example, the external display may display live video footage from imaging device 130, with display configuration 182 shown as an inset. Display configuration 182 provides visual feedback to the user regarding various operational parameters of the device.

Display configuration 182 may include multiple indicators, each displaying specific information about the status and operation of medical device 100. The order and position of each indicator may vary based on user preferences and display size. Display configuration 182 may include a first indicator 184, a second indicator 186, and a third indicator 188.

First indicator 184 may include a first position indicator that represents the position of articulation portion 105. First indicator 184 may include a label (e.g., "P" for "Position") and a visual representation divided into four quadrants. In these aspects, first indicator 184 may be a quadrant-based representation. Each quadrant represents a different directional position of distal tip 128 relative to a longitudinal axis of shaft 104 when it is in a neutral (non-articulated) configuration. Within these quadrants, circles or other shapes may appear. A first symbol 190 (e.g., a solid shape) may indicate a current position of distal tip 128, while a second symbol 192 (e.g., a hollow shape) may indicate a previously marked position that the user flagged for later reference, or a previous position of articulation portion 105. Medical device 100 may be configured to automatically return to this marked position when needed. As distal tip 128 articulates, first symbol 190 moves accordingly within and between the quadrants. For example, in FIG. 1D, first position indicator 184 shows that distal tip 128 is currently articulated downward and rightward relative to the shaft's longitudinal axis, while a previously flagged position is located downward and leftward.

First symbol 190 and second symbol 192 may appear anywhere within the position indicator, including positions that straddle the dividing lines between quadrants. When first symbol 190 or second symbol 192 straddles a dividing line, this indicates that distal tip 128 is in a single-direction articulation position (e.g., purely left, right, up, or down) rather than a combined directional position (such as left-and-up, left-and-down, right-and-up, or right-and-down).

Second indicator 186 may include a second position indicator that represents a position of elevator 134. Second indicator 186 may include a label (e.g., “E” for “Elevator”) and a visual representation comprising a vertical line and a horizontal line. The vertical line may represent a range of angles elevator 134 may achieve, and the horizontal line may represent an approximate angle of the current position of elevator 134. For example, when the horizontal line is at or near a top of the vertical line (e.g., near the label), this may be indicative that elevator 134 is completely open/lowered (e.g., at an angle of approximately 180 degrees relative to the longitudinal axis of shaft 104). When the horizontal line is at or near a bottom of the vertical line (e.g., opposite the label), this may be indicative that elevator 134 is completely closed/raised (e.g., at an angle of approximately 90 degrees or less relative to the longitudinal axis of shaft 104). For example, in FIG. 1D, second position indicator 186 shows that elevator 134 is at an angle of approximately 130 degrees relative to the longitudinal axis of shaft 104. This may indicate that the elevator is partially closed or open. In other aspects, the horizontal line at or near the top of the vertical line may be indicative that elevator 134 is in a completely closed/raised position, and the horizontal line at or near the bottom of the vertical line may indicate that the elevator is in a completely open/lowered position. Accordingly, the illustration of second indicator is exemplary.

Third indicator 188 may include a speed indicator that represents information about the rate of articulation or actuation of aspects of medical device 100, such as the speed of articulation of distal tip 128 or the rate of adjustment of elevator 134. Speed indicator 188 may include a label (e.g., “S” for “Speed” and a bar that raises or lowers as the speed is adjusted. For example, in FIG. 1D, speed indicator 188 shows that the speed of one or both articulation of distal tip 128 and rate of adjustment of elevator 134 is near a maximum speed. Speed indicator 188 may also include multiple bars or be broken into separate bar graphs, for example, to separately represent the rate of articulation of distal tip 128 and the rate of adjustment of elevator 134.

Display 124, including the arrangement of these indicators within display configuration 182 may allow the user to quickly and easily interpret multiple parameters simultaneously during use of medical device 100. This integrated visual feedback system may enhance the user's ability to precisely control and navigate medical device 100 during procedures, potentially improving efficiency and accuracy.

In other aspects, display 124 may be omitted from medical device 100. In these aspects, display configuration 182 may be provided on an external screen or monitor, as discussed above.

Handle 102 may contain a micro-controller 180 that electronically connects to one or more actuators 116, 117, 118, or motors 150, 170, 172. Micro-controller 180 may provide various enhanced functionalities, including positional memory capabilities that allow medical device 100 to store and return to specific configurations of articulation portion 105 or elevator 134, potentially improving procedural efficiency. In some aspects, all or some of the capabilities of micro-controller 180, described below, may be stored on external controller, an edge device, or the cloud.

For example, micro-controller 180 may include pre-programmed procedural routines such as tunneling, canulating, relaxing elevator 134 to facilitate accessory device passage, and creating vibration or "jittering" movements to free snagged accessory devices. These automated routines can reduce procedure duration and minimize operator fatigue. Camera stabilization algorithms within micro-controller 180 may compensate for minor movements and vibrations, enabling operators to obtain clearer images and potentially improving diagnostic capabilities.

The force feedback algorithms housed in micro-controller 180 process sensor data to dynamically adjust device parameters including speed and articulation angles. These adjustments enhance both safety and precision during medical procedures. In some aspects, micro-controller 180 may incorporate pre-programmed articulation limits that prevent over-articulation of articulation portion 105, protecting both the device from damage and enhancing patient safety during procedures.

Micro-controller 180 provides advanced positional holding capabilities that enable operators to maintain specific configurations of articulation portion 105 or elevator 134 without requiring continuous manual pressure on the actuators. This feature significantly reduces operator fatigue during extended procedures by eliminating the need for constant hand engagement with the controls. When an operator achieves a desired position of articulation portion 105 or a specific angle of elevator 134, micro-controller 180 can electronically lock this configuration until the operator deliberately initiates a change. Alternatively, micro-controller 180 can be programmed with a safety return function that automatically returns articulation portion 105 to a straight, unarticulated position or elevator 134 to a neutral, unactuated position when the operator releases the respective actuators. This automatic return capability provides a fail-safe mechanism that helps prevent unintended movement of the medical device within the patient's anatomy when operator input ceases.

In some aspects, advanced implementations of micro-controller 180 may incorporate sophisticated machine learning or Artificial Intelligence (AI) capabilities that significantly enhance procedural precision and efficiency. For example, once a target tissue is identified during a procedure, proprietary computer vision algorithms can automatically analyze the visual field and adjust articulation portion 105 or elevator 134 to stabilize the image on that specific tissue region. These algorithms employ real-time image processing techniques to identify distinctive tissue features, track their position within the field of view, and maintain optimal visualization despite patient movement or breathing. If the scope moves while this feature is enabled, micro-controller 180 can direct motors 150, 170, 172 to compensate for the movement through a predictive motion model, maintaining the target tissue in focus with minimal latency. This adaptive targeting system can be particularly valuable during extended procedures where maintaining consistent visualization of specific anatomical structures is critical for diagnostic accuracy or therapeutic intervention. The AI capabilities may also include procedural pattern recognition, where micro-controller 180 learns from operator behaviors across multiple procedures to anticipate common articulation sequences and optimize response characteristics accordingly, potentially reducing procedure duration and operator fatigue.

Micro-controller 180 may collect comprehensive operational data during medical procedures, including but not limited to procedure duration, frequency and extent of articulation portion 105 movements, elevator 134 positioning sequences, movement speeds across different tissue environments, tissue interaction forces measured through integrated sensors, user control patterns, and routine usage statistics. This extensive data collection creates valuable datasets that can be analyzed to identify optimal procedural techniques, establish benchmarks for different types of procedures, and inform the development of future robotic surgical applications. The accumulated data may be used to enhance micro-controller algorithms through machine learning techniques, enabling adaptive control systems that anticipate user needs based on procedural context. Over time, this continuous improvement cycle may significantly enhance device performance by optimizing response characteristics, reducing procedure duration, minimizing tissue trauma, and improving overall procedural outcomes. Additionally, the collected data may provide insights for training new operators and developing simulation systems that accurately replicate real-world usage scenarios, potentially accelerating the learning curve for complex procedures. In some aspects, the data collected may be transmitted to an external controller, edge device, or cloud.

In some aspects, motors 150, 170 ,172 may be configured to be selectively activated or deactivated. This feature may allow users to customize the operational feel of the medical device according to their preferences or specific procedural requirements. For example, if a user desires a more traditional or mechanical feel during operation, the motors may be turned off, reverting the device to a manual control mode. In such aspects, medical device 100 may include controls (e.g., knobs) that may facilitate such manual control. Such knobs may be used in place of a joy-stick style actuator as shown for first actuator 116. For example, one knob may be coupled to each of first pulley 140 and second pulley 142. One or more knobs may include ring motors. This option may be particularly useful for operators who are more accustomed to conventional mechanical devices or in situations where direct manual control is preferred.

Alternatively or additionally, micro-controller 180 may incorporate algorithms designed to simulate a more mechanical feeling for the user, even when the motors are active. These algorithms may adjust motor response characteristics, introduce controlled resistance, or modify feedback mechanisms to emulate the tactile sensations typically associated with mechanical systems. This approach may provide users with the familiarity of traditional mechanical controls while still benefiting from the precision and advanced capabilities of the motorized system.

The ability to switch between motorized and manual modes, or to adjust the feel of the motorized system, may enhance the versatility of the medical device. It may allow for a smoother transition for users adapting to the new technology and provide flexibility in various clinical scenarios where different levels of assistance or control may be desired.

Finally, handle 102 may include a battery to assist with powering micro-controller 180 or motors 150, 170, 172. The integration of a battery within handle 102 may provide for increased portability and reduced reliance on external power sources. In some aspects, the battery may provide enough power to enable the wireless data transmission of data from micro-controller 180 to an external monitor, an external controller, an edge device, or the cloud.

During use of medical device 100, one or more of circuit board 146, micro-controller 180, or an internal or external computer or controller may be configured to receive information related to a position of articulation portion 105 of medical device 100. The position of articulation portion 105 may be displayed on user interface or display 124. As discussed above, user interface or display 124 includes a first indicator 184 that may be a quadrant illustration and may include first symbol 190 representing a current position of articulation portion 105, and second symbol 192 representing a previous position of articulation portion 105. In some aspects, medical device 100 may be configured to automatically move articulation portion 105 from a current position to the previous position.

For example, one or more of circuit board 146, micro-controller 180, or an internal or external computer or controller may be configured to receive an input from the user or from the one or more force sensors discussed above. Upon receiving the input, one or both motors 170, 172 may rotate to automatically adjust a position of articulation portion 105. In some aspects, articulation portion 105 may be moved from a current position to the previously marked position. In other aspects, articulation portion 105 may be moved from the current position to a neutral (un-articulated) position.

Additionally or alternatively, one or more of circuit board 146, micro-controller 180, or an internal or external computer or controller may be configured to receive information related to a position of elevator 134 of medical device 100. As discussed above, the position of elevator 134 may be displayed on user interface or display 124. In some aspects, medical device 100 may be configured to automatically move elevator 134 from a current position to a previous position, a fully open position, or a fully closed position. For example, one or more of circuit board 146, micro-controller 180, or an internal or external computer or controller may be configured to receive an input from the user or from the one or more force sensors discussed above. Upon receiving the input, motor 150 may be configured to automatically adjust a position of elevator 134.

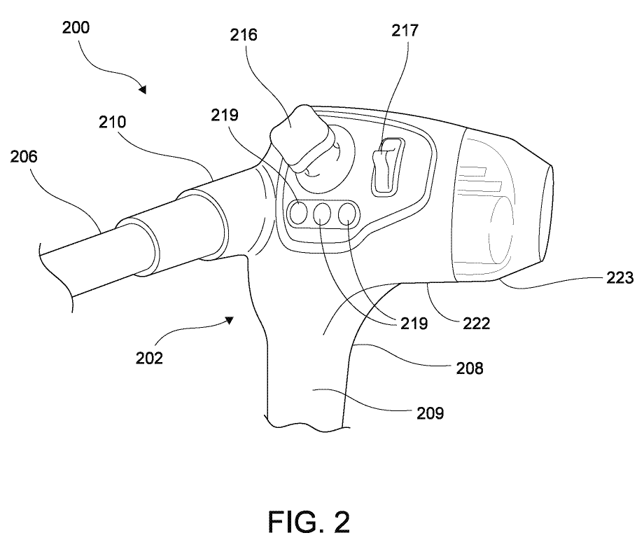

Referring to FIG. 2, an alternative handle configuration of a medical device 200 is illustrated. Medical device 200 may have any or all of the same characteristics of medical device 100, except as described below. For example, medical device 200 includes a handle 202 with a gripping portion 208 designed for comfortable and secure handling during use. Handle 202 may include one or more handle housing pieces 209 configured to contain internal mechanisms of handle 202. Handle 202 may be used in conjunction with shaft 104, including articulation portion 105, distal tip 128, imaging device 130, illumination device 132, elevator 134 of medical device 100. Medical device 200 may further include an umbilicus 206 connected to handle 202 via a port 210.

Handle 202 features a first actuator 216, a second actuator 217 or one or more third actuators 219. These actuators may be arranged in a configuration that allows for ergonomic access and efficient operation of medical device 200. For example, a positioning of these actuators may enable the user to control multiple functions of the medical device 200 with minimal hand movement.

First actuator 216 may have any or all of the same characteristics as first actuator 116 of medical device 100. For example, first actuator 216 may be a joystick configured to control a relative position of articulation portion 105 (FIG. 1A).

Second actuator 217 may have any or all of the same characteristics as second actuator 117 of medical device 100. For example, second actuator 217 may be a slider, lever, or knob configured to control a relative position of elevator 134 (FIG. 1A).

Third actuators 219 may include a plurality of actuators, including one or more buttons, switches, etc. Each third actuator 219 may be configured to accomplish one or more tasks of medical device 200, including, but not limited to locking a position of articulation portion 105 or elevator 134, flagging a position of articulation portion 105 or elevator 134, image capture, video capture, irrigation, insufflation, etc. In some aspects, at least one third actuator 219 may be configured to reset a position of articulation portion 105 or elevator 134 to a neutral configuration. Each third actuator 219 may be pre-programed or may be programmable by a user.

An extension 222 protrudes from the main body of the handle 202. This extension may provide additional control functionality or serve as a mounting point for accessories.

Medical device 200 may include a housing cover 223 that encloses the internal components, protecting them from damage and providing a smooth exterior surface. In aspects, housing cover 223 may be at least partially transparent to enable a user to observe the inner workings of handle 202. Housing cover 223 may be removable to enable a user to replace internal components, as necessary.

Although medical device 200 may not include an integrated display (such as display 124 of medical device 100), a user interface, such as display configuration 182 described above with respect to FIG. 1D, may be displayed on an external monitor. For example, display configuration 182 may be overlayed with a live image or may be displayed as an inset (e.g., picture-in-picture display). This display configuration may enhance the user's ability to precisely control and navigate medical device 100 during procedures, potentially improving efficiency and accuracy.

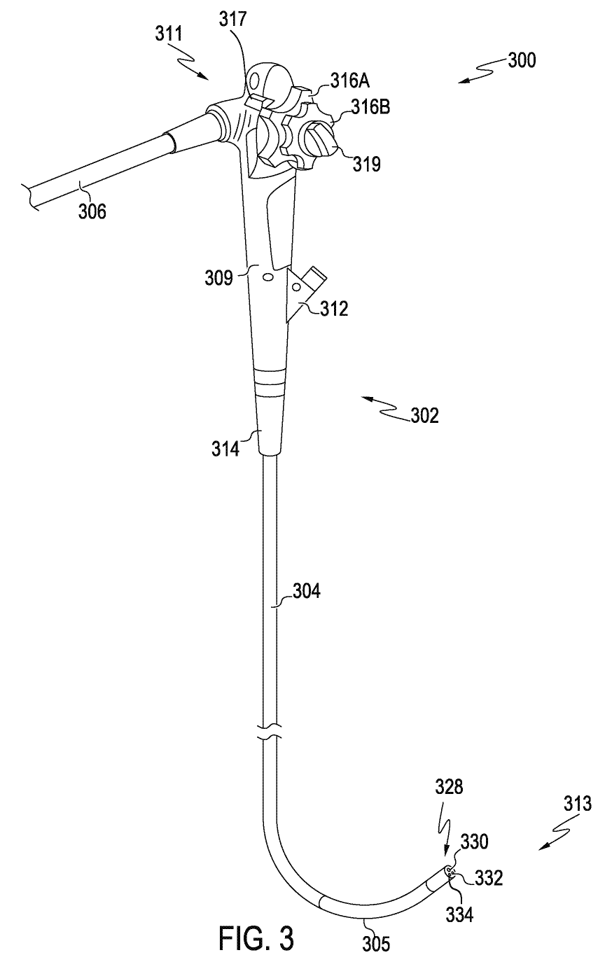

FIG. 3 illustrates an exemplary medical device 300 (e.g. an endoscope or duodenoscope) in accordance with an embodiment of this disclosure. Medical device 300 may have any or all of the characteristics of medical device 100 or medical device 200, described above, except as described below. For example, medical device 300 includes a proximal end 311 and a distal end 313. The proximal end 311 of medical device 300 may include a handle 302 having one or more actuators, such as knobs 316A, 316B, actuators 317, or locks 319. The actuators may be any suitable type of actuator, such as knobs, levers, sliders, joysticks, or the like, unless otherwise stated below. Handle 302 may also include one or more ports 312 for inserting instruments into a working channel of medical device 300. Handle 302 may have a body defined by one or more handle housing pieces 309 that form a body of handle 302 and enclose elements of handle 302. An umbilicus 306 may extend away from proximal end 311 of medical device 300.

A shaft 304 extends from a distal end of handle 302 to distal end 313 of medical device 300. Shaft 304 may be configured for insertion into a body lumen of a subject. A strain relief 314 may be positioned at the junction of handle 302 and shaft 304. Strain relief 314 may be designed to reduce stress concentration and prevent kinking or damage to internal components at this transition point. At least a portion of shaft 304 may be flexible, semi-flexible, or rigid.

A distal end portion of shaft 304 may include an articulation portion 305 and a distal tip 328 that is fixed to a distal end of articulation portion 305. In some aspects, articulation portion 305 may include an articulation joint that is articulated or steered via controls (e.g., actuators, such as actuators 316A, 316B) on handle 302, allowing a user to maneuver distal tip 328 within the body. For example, articulation portion 305 may be configured to bend in one or more directions relative to a longitudinal axis of more proximal portions of shaft 304 (e.g., left, right, up, or down).

Described in further detail below, a force required to articulate articulation portion 305 may be changed using motors assemblies disposed within handle 302. Such motor assemblies may be activated, partially activated, or deactivated, depending on a user’s preferences. Additionally or alternatively, the motor assemblies described herein may be configured to change the force required to actuate the elevator or other aspects of medical device 300.

Distal tip 328 may include an imaging device 330 and an illumination device 332. Imaging device 330 may be side-facing (as shown in FIG. 1A) or forward-/distal-facing (as shown in FIG. 3). Illumination device 332 may include one or more light sources, such as light-emitting diodes (LEDs) or optical fibers. Although not shown, distal tip 328 may further include an elevator (similar to elevator 134 of FIG. 1A) that is configured to change the angle or direction of instruments or tools that are passed through medical device 300. For example, an instrument or tool may be inserted through port 312 of handle 302 that is in communication with a lumen or a working channel of shaft 304. The instrument or tool may be extended through the lumen or working channel and out of a distal opening of distal tip 328. In some aspects, the elevator may be configured to enable the user to adjust the position or orientation of instruments exiting the distal tip 328 during a procedure. Distal tip 328 may further include additional openings 334 to facilitate the insertion or removal of tools, objects, or fluids, into the body.

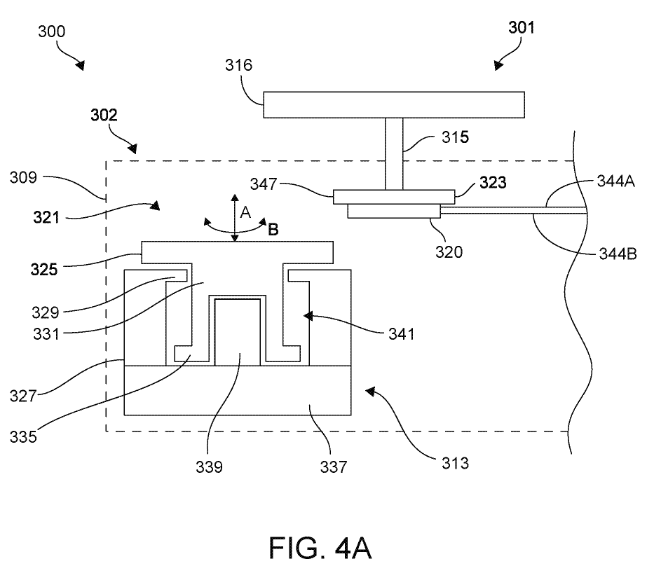

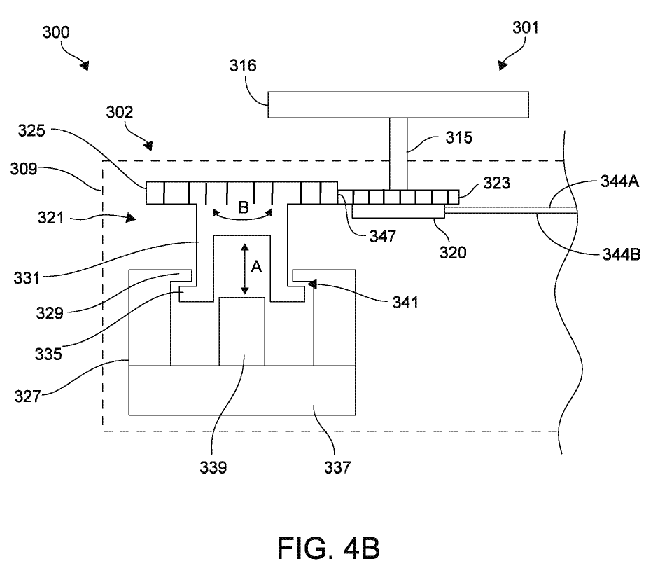

FIGS. 4A and 4B each illustrate a schematic of an exemplary motor assembly 321 disposed within a portion of handle 302 (e.g., within housing pieces 309) of medical device 300. FIG. 4A illustrates motor assembly 321 in an inactivated or disengaged state, and FIG. 4B illustrates motor assembly 321 in an activated or engaged state. Each state of motor assembly 321 will be described in further detail below. For example, motor assembly 321 may be configured to interact with a knob assembly 301 to rotate an actuator, such as knob 316, with or without user input, or to reduce a force required for a user to rotate the knob.

Motor assembly 321 may be contained within handle housing pieces 309 of medical device 300. Handle housing pieces 309 of handle 302 are shown in broken lines in FIGS. 4A and 4B for clarity of illustration, however, the exact configuration of motor assembly 321 may vary within handle 302.

Referring to both FIGS. 4A and 4B, medical device 300 may include a knob assembly 301 and a motor assembly 321. Knob assembly 301 may be comprised of an actuator or a knob 316 fixedly coupled to a knob gear 323 by an axle 315. A pulley 320 may be fixed to knob gear 323 such that, when actuator 316 is rotated, axle 315, knob gear 323, and pulley 320 are rotated accordingly. Axle 315 may extend through an opening of handle housing piece 309, such that axle 315 is partially within handle 302 and partially outside of handle 302. One or more steering wires 344A, 344B may be coupled to pulley 320. Although steering wires 344A, 344B are referred to herein, it will be appreciated that a wire coupled to pulley 320 may have other functions, such as actuation of an elevator. Accordingly, when actuator 316 is rotated, each of steering wires 344A, 344B may be moved (e.g., translated) axially, proximally or distally. When steering wires 344A, 344B are moved proximally or distally, articulation portion 305 (FIG. 3) may be articulated in one or more directions, because distal ends of wires 344A, 344B may be coupled to articulation portion 305.

Motor assembly 321 may be comprised of a motor 337, a solenoid 327, and a motor gear 331. Solenoid 327 may at least partially surround a portion of motor gear 331. In other words, motor gear 331 may extend into a lumen defined by windings of solenoid 327. Motor gear 331 may be configured to linearly translate towards and away from knob assembly 301 (as indicated using arrow “A” in FIGS. 4A and 4B) and rotate in a first direction or second direction (indicated using arrow “B” in FIGS. 4A and 4B) within solenoid 327. In aspects, solenoid 327 may include a flange or shoulder 329 protecting radially inward, relative to a central axis of solenoid 327, from an end of solenoid 327 that is closer to actuator 316. Shoulder 329 may be configured to interact with a flange or arm 335 projecting radially outward (relative to a central axis of solenoid 327) from an end of motor gear 331 that is further from actuator 316. For example, shoulder 329 may be configured to interact with arm 335 to prevent movement of gear 331 linearly (in direction A) past shoulder 329 (e.g., in an actuated state, as shown in FIG. 4B), while still permitting rotational movement of motor gear 331 within solenoid 327.

FIGS. 4A and 4B illustrate a single knob assembly 301 and a single motor assembly 321 for ease of illustration and clarity. However, it may be understood that medical device 300 may include multiple knob assemblies 301 and multiple motor assemblies 321 to provide comprehensive control over various functions of the device. In some aspects, each knob assembly 301 may be paired with a corresponding motor assembly 321 to enable independent control of different articulation directions or device functions. For example, one knob assembly 301 and motor assembly 321 pair may control up-down articulation of articulation portion 305 (FIG. 3), while another knob assembly 301 and motor assembly 321 may control left-right articulation of articulation portion 305 (FIG. 3). Additional knob assemblies 301 and motor assemblies 321 may be incorporated to control other functions such as elevator positioning, rotation of distal tip 328, or actuation of a lock, or other actuatable components of medical device 300.

Motor 337 may be configured to provide power to solenoid 327. Alternately solenoid 327 may be configured to provide power to motor 337. Alternatively, both may be powered separately. Motor 337 may further include a shaft 339 extending at least partially into a portion of motor gear 331. In some aspects, shaft 339 may be configured to maintain a relative position of motor gear 331 within solenoid 327 in both the actuated and unactuated states. Shaft 339 may couple motor 337 to motor gear 331 (e.g., via a spline or other axial translating interface that will maintain alignment and transmit torque). For example, shaft 339 may be received within a lumen of motor gear 331, such that motor gear 331 is keyed to motor 337. Shaft 339 may have one or more slots or protrusions, as discussed below, to facilitate rotation of motor gear 331 upon activation of motor 337. This configuration may allow motor gear 331 to remain properly aligned within solenoid 327 and provide rotation of motor gear 331 regardless of the operational state of motor assembly 321.

In the first, disengaged state or lowered position of motor assembly 321, as shown in FIG. 4A, toothed gear 325 of motor gear 331 may be axially misaligned (along an axial direction of shaft 339) with toothed gear 347 of knob gear 323. Accordingly, a gap 341 may be defined between shoulder 329 and arm 335. Accordingly, motor gear 331 may be at a first height relative to motor 337 in the unactuated state. In the unactuated state, motor assembly 321 may not be in contact with knob assembly 301 such that the user may have full, unassisted control of actuator 316. For example, when motor assembly 321 is in this disengaged state, rotation of actuator 316 may directly translate to movement of steering wires 344A, 344B without any interference or assistance from motor assembly 321. This configuration may allow for precise manual control of articulation portion 305, which may be desirable in certain procedural situations or based on user preference.

In the second, engaged state of motor assembly 321, as shown in FIG. 4B, toothed gear 325 and toothed gear 347 may be axially aligned. Thus, a size of gap 341 between shoulder 329 and arm 335 may decrease, or gap 341 may be absent. Accordingly, motor gear 331 may be at a second height relative to motor 337. The second height may be greater than the first height. In the actuated state, motor assembly 321 may be in contact with knob assembly 301.

To transition from the first, disengaged state to the second, engaged, state, a current may be provided to solenoid 327, such that a current flows through solenoid 327. In some aspects, solenoid 327 may be powered by motor 337 or provide power to the motor 337. In other aspects, both motor 337 and solenoid 327 may be provided power by an alternative source. When solenoid 327 is powered by motor 337, turning motor 337 on causes transition to the engaged state and causes motor 337 to assist rotation of knob assembly 301. When solenoid 327 is powered by another source, motor 337 may be continuously on, or a state of motor 337 may not directly determine whether motor 337 assists rotation of knob assembly 301. A flow of current through solenoid 327 may generate a magnetic field, which may interact with motor gear 331 (e.g., motor gear 331 may include ferromagnetic material) causing motor gear 331 to move upwards, or towards knob assembly 301.

When motor assembly 321 is in the engaged state, motor gear 331 may interact with knob gear 323, such that rotation of motor gear 331 causes rotation of knob gear 323. An outer edge or perimeter of toothed gear 325 of motor gear 331 may include teeth or other features (shown schematically in FIG. 4B) that interact with complementary teeth or complementary features disposed on an outer edge or perimeter of toothed gear 347 of knob gear 323 of knob assembly 301. This engagement may allow for the transfer of rotational force from motor gear 331 to knob gear 323. For example, when motor gear 331 is rotated, knob gear 323 may be simultaneously rotated. In some aspects, these features may be configured to allow for easy engagement and disengagement as solenoid 327 moves motor gear 331 between its upper and lower positions.

In some aspects, knob gear 323 may be omitted. For example, axle 315 may extend between and couple actuator 316 directly to pulley 320. In such a configuration, motor gear 331 may be configured to interact directly with pulley 320. For example, an outer edge or perimeter of pulley 320 may include complementary features configured to engage with the teeth or features disposed about the perimeter pf toothed gear 325 of motor gear 331.

When motor assembly 321 is in this engaged state or upper position, rotation of motor gear 331 may be translated to knob gear 323, providing motorized assistance or control to the movement of steering wires 344A, 344B or other actuation elements (e.g., wires). This configuration may allow for precise motorized control of articulation portion 305 (or other aspects, such as an elevator). In some aspects, this configuration may reduce the forces required to rotate knob 316 and articulate articulation portion 305. For example, engagement between motor gear 331 and knob gear 323 may provide a hybrid control system, where both manual input through actuator 316 and motorized input through motor 337 can influence the movement of steering wires 344A, 344B. In some aspects, motor assembly 321 may entirely control movement of steering wires 344A, 344B. In other aspects, motor assembly 321 may assist a user in manually actuating actuator 316.

Additionally or alternatively, motor assembly 321 may be configured as a locking mechanism, or a stopping mechanism, for knob 316. For example, when motor assembly 321 is engaged with knob assembly 301, motor assembly 321 may be configured to maintain a rotational position of knob 316. Accordingly, when a user releases knob 316, the position of knob 316 may be maintained via motor assembly 321.

Motor 337 may be controlled by a microprocessor or other control system within medical device 300, allowing for programmed movements or responses to user inputs. In some cases, the control system may modulate the power supplied to motor 337, allowing for variable levels of assistance or control in the engaged state. For example, increasing power to motor 337, may result in a user experiencing decreased forces on knob 316. Conversely, decreasing power to motor 337, thus decreasing power to motor gear 331, may result in a user experiencing increased forces on knob 316. The hybrid manual/motor drive system provided by motor assembly 321 may offer users the flexibility to switch between manual and motorized control as needed during a procedure.

In an alternate embodiment, a single self-restoring three-position solenoid may cause motor gear 331 to travel in a first direction to engage with knob assembly 301A or travel in a second direction to engage with knob assembly 301B when not sitting in a neutral/disengaged position. This may reduce the number of motors necessary to drive the system.

In some aspects, medical device 300 may include a mechanism for the user to switch between engaged and disengaged states. This mechanism may be a physical switch on handle 302, or it may be controlled through a user interface of medical device 300, such as internal or external display. The ability to switch between states may provide flexibility during procedures, allowing the user to choose between manual and motorized control as needed.

In some aspects, motor assembly 321 may include additional components such as sensors or encoders to detect the position or rotation of knob assembly 301 and/or the forces acting upon knob 316. This information may be used by a control system to precisely control the articulation of articulation portion 305 (FIG. 3) or the actuation of other components of medical device 300. Furthermore, this information may be displayed on a user interface (e.g., an internal or external display).

In some aspects, motor assembly 321 may be configured to switch between multiple states. For example, in a first state, as shown and previously described with reference to FIG. 4A, knob assembly 301 may be in a fully manual mode. In this state, motor assembly 321 may be completely disengaged from knob assembly, allowing the user to have direct mechanical control over the articulation portion 305 and other actuatable components via knob 316. This state may provide the user with traditional tactile feedback and direct force transmission, which may be preferred during certain phases of a procedure where immediate manual response is desired.

A second, third, or fourth state of motor assembly 321 are illustrated in FIG. 4B. For example, in the second state, motor assembly 321 may operate in a motor-assisted mode. As shown in FIG. 4B, in this configuration, motor assembly 321 may be engaged with knob assembly 301 to provide assistance to rotate knob 316 while still allowing the user to maintain primary control. For example, motor assembly 321 may be positioned to mesh with the knob assembly 301. In this second state, motor assembly 321 at a reduced power level or with variable assistance based on user input force. This state may reduce the physical effort required by the user to rotate knob 316 while preserving tactile feedback and some manual control characteristics. The assistance level may be adjustable, for example, by adjusting the power levels of motor assembly 321, allowing users to customize the amount of motorized support based on their preferences and the specific requirements of the procedure.

In the third state, motor assembly 321 may operate in a fully motorized mode. In this state, motor assembly 321 may take primary control of the articulation and actuation functions of medical device 300, with user inputs serving as command signals rather than direct mechanical forces. For example, articulation and actuation of medical device 300 may be controlled by the user interface. Inputs may be supplied via the user interface and transmitted to motor assembly 321. Accordingly, a user may not need to touch or rotate knob 316 but may instead control rotation of knob 316 via the user interface. For example, motor assembly 321 may be fully engaged with knob assembly 301. This state may provide the highest level of precision and consistency, which may be beneficial for delicate procedures requiring fine control or when fatigue reduction is a priority. In such a state, motor assembly 321 may be configured to operate according to programmed parameters, potentially incorporating feedback from sensors to maintain desired positions or movements.

In the fourth state, motor assembly 321 may operate in an autonomous or semi-autonomous mode. In this configuration, the motors may operate based on pre-programmed routines, sensor feedback, or artificial intelligence algorithms with minimal direct user input. The system may automatically adjust articulation portion 305 or other components based on procedural requirements, environmental conditions, or safety parameters. This state may be particularly useful for repetitive tasks, stabilization functions, or complex maneuvers that benefit from automated precision. The user may retain override capabilities to intervene or adjust the automated functions as needed during the procedure.

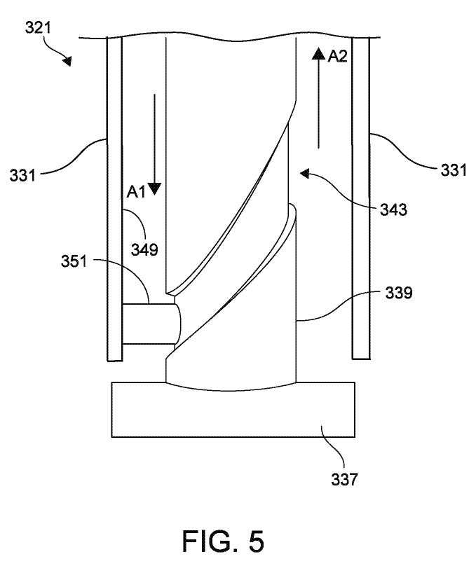

FIG. 5 illustrates an exemplary configuration of shaft 339 of motor assembly 321 of FIGS. 4A and 4B. As shown in FIG. 5, shaft 339 may include a channel or helical groove 343 that extends along at least a portion of its length. Helical groove 343 may enable both rotational and linear (illustrated using arrows labeled “A1” and “A2”) movement capabilities of motor gear 331 (FIGS. 4A and 4B) within motor assembly 321. For example, motor gear 331 may include one or more guide pins or projections 351 that extend radially inward from an internal surface 349 of motor gear 331 and interact with helical groove 343 of shaft 339. In alternatives, shaft 339 may include projection 351, and motor gear 331 may include channel or helical groove 343.

When solenoid 327 (FIGS. 4A and 4B) is activated, motor gear 331 may move axially relative to shaft 339, as described above. Helical groove 343 may guide the movement of guide projection(s) 351, maintaining engagement between projection 351 and groove 343. When motor 337 is activated, projection 351 may remain engaged with helical groove 343 (e.g., at a bottom of helical groove 343 when motor gear 331 is engaged with knob gear 323 in the engaged position). In some aspects, motor 337 may also cause rotation of motor gear 331 even in the disengaged state. Solenoid 327 controls an axial position of motor gear 331, while motor 337 controls rotation of motor gear 331.

Although a single projection 351 is shown in FIG. 5, motor gear 331 may include multiple guide projections 351 that interact with corresponding grooves on motor shaft 339. This configuration may increase the load-bearing capacity of the mechanism and provide more stable and precise movement. In some aspects, the keyed configuration may also serve as a safety feature or a stop. For example, the design of the helical groove may limit the range of motion of motor gear 331, preventing over-rotation or over-extension that could potentially damage the device or cause unintended movements of articulation portion 305.

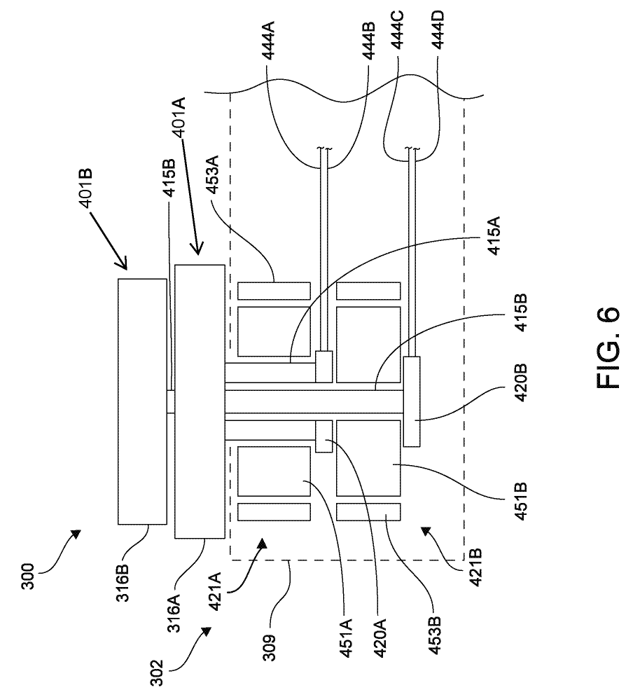

FIG. 6 illustrates an alternative configuration of one or more motor assemblies disposed within medical device 300 of FIG. 3. For example, FIG. 6 illustrates a first motor assembly 421A configured to interact with a first knob assembly 401A, and a second motor assembly 421B configured to interact with a second knob assembly 401B. Each of first knob assembly 401A and second knob assembly 401B may have any or all of the same characteristics as knobs described above, except as described below.

In particular, first knob assembly 401A may include a first knob 316A coupled to a first pulley 420A via a fist axle 415A. Second knob assembly 401B may include a second knob 316B coupled to a second pulley 420B via a second axle 415B. Second axle 415B may extend through first knob 316A, first axle 415A and first pulley 420A. In these aspects, second axle 415B may be concentric and coaxial with first axle 415A.

First motor assembly 421A may be configured to interact with first knob assembly 401A to rotate first knob 316A with or without user input, or to reduce a force required for a user to rotate first knob 316A. Similarly, second motor assembly 421B may be configured to interact with second knob assembly 401B to rotate second knob 316B with or without user input, or to reduce a force required for a user to rotate second knob 316B. For example, when first knob 316A is rotated, first axle 415A and first pulley 420A are rotated accordingly. Rotation of first pulley 420A may result in the linear movement (tensioning or detensioning) of a first control wire 444A and a second control wire 444B, each coupled to first pulley 420A at a first end. A second, opposite end of each first control wire 444A and second control wire 444B may be coupled to articulation portion 305 (FIG. 3), thus resulting in the articulation of articulation portion 305 in a first direction or a second direction.

Similarly, when second knob 316B is rotated, second axle 415B and second pulley 420B are rotated accordingly. Rotation of second pulley 420B results in the linear movement of a third control wire 444C and a fourth control wire 444D, each coupled to second pulley 420B at a first end. A second, opposite end of each third control wire 444C and fourth control wire 444D may be coupled to articulation portion 305 (FIG. 3), thus resulting in the articulation of articulation portion 305 in a third direction or a fourth direction.

First motor assembly 421A may include a first rotor 451A configured to interact with an outer surface of first axle 415A, and a first stator 453A configured to interact with an outer surface of first rotor 451A. Rotor 451A may be a rotating component of an electric motor that converts electrical energy into mechanical rotational motion. Rotor 451A may include magnetic elements, windings, or other components that interact with magnetic fields to produce rotation when energized. Stator 453A may be a stationary component of an electric motor that generates a magnetic field to interact with rotor 451A. Stator 453A may include electromagnetic windings, permanent magnets, or other magnetic field-generating elements arranged to create the magnetic field necessary for motor operation. Rotor 451A and stator 453A may have any of the features of rotors and stators known in the art.

First rotor 451A may be configured to assist with or facilitate the rotation of first axle 415A, thus facilitating the rotation of first pulley 420A. For example, when first stator 453A is energized, it may generate a magnetic field that interacts with first rotor 451A, causing first rotor 451A to rotate. This rotation may be transmitted to first axle 415A through direct contact or mechanical coupling between the rotor and the outer surface of the axle. For example, first stator 453A may be directly coupled to first axle 415A or first pulley 420A. Although a gap is shown between first stator 453A and these components to illustrate the separate elements of medical device 300, first stator 453A may contact and be coupled to first axle 415A or first pulley 420A. The rotational assistance provided by first rotor 451A may reduce or eliminate the mechanical effort required from the user when manipulating first knob 316A. Accordingly, the force required to rotate first knob 316A may be reduced.

Similarly, second motor assembly 421B may include a second rotor 451B (having any property of first rotor 451A) configured to contact an outer surface of second axle 415B or second pulley 420B and a second stator 453B (having any property of first stator 453A) configured to interact with an outer surface of second rotor 451B. Second rotor 451B may be configured to assist with or facilitate the rotation of second axle 415B, thus facilitating the rotation of second pulley 420B. For example, when second stator 453B is energized by a same motor as first stator 453A or a second, different motor, it may generate a magnetic field that interacts with second rotor 451B, causing the rotor to rotate. This rotation may be transmitted to second axle 415B through direct contact or mechanical coupling between the rotor and the outer surface of the axle, as discussed above. The rotational assistance provided by second rotor 451B may reduce or eliminate the mechanical effort required from the user when manipulating second knob 316B. Accordingly, the force required to rotate second knob 316B may be reduced, potentially improving user comfort and control precision during medical procedures.

Each motor assembly 421A and 421B may be operated independently of one another, allowing for customized control of different articulation directions. This independent operation may enable users to select different operational modes for each knob, thus providing different operational modes for each axis of articulation of articulation portion 305 (FIG. 3). For example, first motor assembly 421A may operate in a fully motorized mode for precise up-down articulation control, while second motor assembly 421B may simultaneously operate in a motor-assisted mode for left-right articulation, providing tactile feedback to the user during these movements.

Each motor assembly 421A and 421B may operate in four different states, similar to motor assembly 321 previously discussed with reference to FIGS. 4A and 4B. These states may provide distinct operational characteristics for each axis of control, allowing users to select the most appropriate control mode for each direction based on procedural requirements and user preferences. For example, each of motor assembly 421A, 421B may maintain the same operational states as previously described motor assemblies, including fully manual, motor-assisted, fully motorized, and autonomous modes. In fully motorized modes, a user may control motor assemblies 421A, 421B using a joystick such as those described above.

FIG. 7 illustrates an alternative configuration of a motor assembly 521 comprised of a single stator 553 that may be configured to operate two separate rotors, such as a first rotor 551A and a second rotor 551B. Unless otherwise, stated, motor assembly 521 may have any property of medical device 300, except as stated herein. Reference numbers ending in the same tens and ones digits refer to like parts, and discussion of analogous parts is omitted except to highlight differences. Single stator 553 may encompass rotors 551A, 551B within a unified magnetic field generation system. This configuration may provide advantages in terms of manufacturing efficiency, space utilization, and magnetic field optimization compared to separate stator arrangements. First rotor 551A may be configured to interact with first axle 515A, thus rotating first pulley 520A. Similarly, second rotor 551B may be configured to interact with second axle 515B, thus rotating second pulley 520B.

Single stator 553 may include a first opening 561A through which a first control wire 544A and a second control wire 544B may extend distally through. Single stator 553 may include a second opening 561B through which a third control wire 561C and a fourth control wire 561D may extend distally through. These openings may be strategically positioned to allow the control wires to pass through the stator assembly without interfering with the magnetic field generation or motor operation. In alternatives, first opening 561 A and second opening 561B may be combined. In such cases, pulleys 520A and 520B may be toward a center of motor assembly 521, facing and adjacent to one another.

Single stator 553 may be configured to permit rotation of a first pulley 520A and a second pulley 520B independently. This independent rotation capability may allow for differential control of articulation directions, where one pulley (e.g., first pulley 520A or second pulley 520B) may rotate at a different speed or in a different direction than the other pulley. Single stator may incorporate magnetic field patterns or winding configurations that enable independent control of each rotor while sharing the common stator structure.

For example, single stator 553 may include electromagnetic windings or permanent magnets arranged to create distinct magnetic field zones for each rotor 551A, 551B. These zones may be electrically isolated or controlled separately to enable independent operation of each rotor 551A, 551B. The magnetic field distribution within single stator 553 may be optimized to provide adequate torque and control precision for both rotors 551A, 551B simultaneously.

Motor assembly 521 may maintain the same operational states as previously described motor assemblies, including fully manual, motor-assisted, fully motorized, and autonomous modes. The single stator configuration may enable these operational modes for both rotors independently or in coordinated fashion, depending on the control requirements and user preferences during medical procedures.

The disclosed medical device systems incorporate hybrid manual-motorized control mechanisms that may provide enhanced precision and reduced user fatigue during medical procedures. The systems may feature motor assemblies that can operate in multiple states, including fully manual, motor-assisted, fully motorized, and autonomous modes, allowing users to select the most appropriate control method based on procedural requirements. The motor assemblies may utilize various configurations, such as ring motors with rotors and stators, solenoid-actuated engagement systems, and single or dual stator arrangements that can control multiple articulation directions independently. These systems may be integrated into compact handle designs that maintain ergonomic functionality while providing advanced control capabilities for articulating distal portions of medical devices such as endoscopes. The hybrid approach may allow for seamless transitions between manual tactile feedback and precise motorized control, potentially improving procedural outcomes through enhanced maneuverability and reduced operator strain during extended medical interventions.

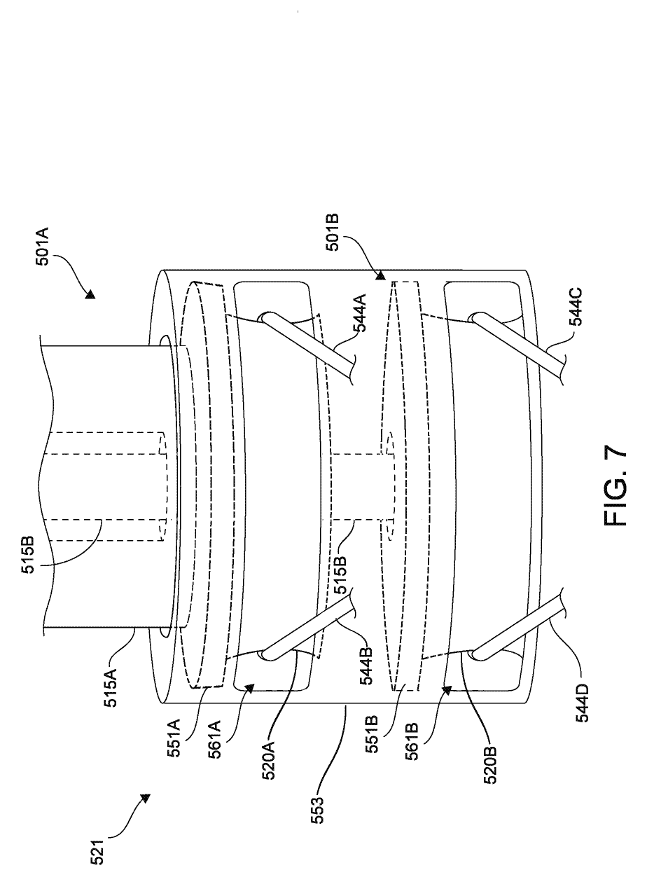

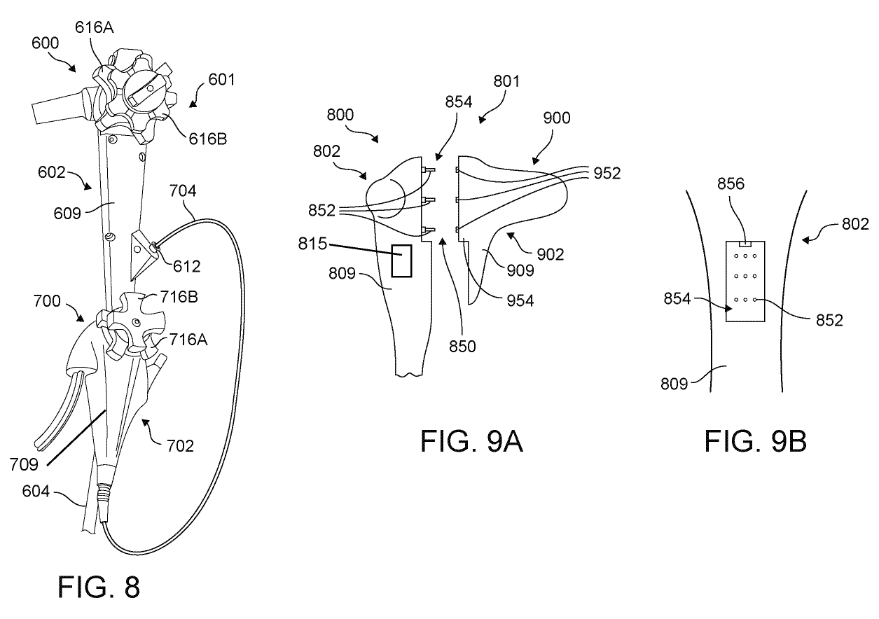

FIG. 8 shows a medical system 601 having a first medical device 600 and a second medical device 700. The medical devices 600 and 700 may have any of the properties of the medical devices described above. In some examples, first medical device 600 may be a duodenoscope or endoscope or other type of “mother” scope. First medical device 600 may have a handle 602 that includes actuators (e.g., knobs) 616A and 616B for steering (articulating) an articulation section (having any of the properties discussed above) of a shaft 604 of first medical device 600. Handle 602 may have a handle body 609. Handle 602 may also include a port 612 that is fluidly coupled to a working channel of first medical device 600.

Second medical device 700 may be, in aspects, a cholangioscope or other type of “daughter” or accessory scope (or other accessory device). Second medical device 700 may include a handle 702 and a shaft 704. Handle 702 may include actuators (e.g., knobs) 716A and 716B. Handle 702 may also include a housing 709. During use, shaft 704 may be inserted into port 612 and advanced distally through the working channel of first medical device 600. A distal tip of shaft 704 may be advanced out of an opening at a distal tip of shaft 704 (e.g., similar to the distal tips described above). Shaft 704 may have an articulation section that may be articulated using 716A and 716B once shaft 704 is extended out of the distal opening of shaft 704.

FIGS. 9A and 9B show aspects of a system 801 having properties of system 601, unless otherwise specified. System 801 may include a first medical device 800 (having any property of first medical device 600) and a second medical device 900 (having any property of second medical device 700). First medical device 800 may have a handle 802 that has actuator(s) of any type described herein (e.g., knobs, joysticks, or the like). Second medical device 900 may have a handle 902 that may also include such actuator(s) or may omit steering actuators.

A housing 809 of handle 802 may be coupled to a housing 909 of handle 902, as described herein. Handle 802 may be coupled to handle 902 via an interface 850. Housing 809 may include one or more connectors 852 that are configured to mate with one or more connectors 952 of housing 909. For example, one or more connectors 852 may include one or more posts, pins, or prongs and one or more connectors 952 may include one or more holes, sockets, or recesses (or vice versa). Other types of connectors (e.g., card connectors, contact connectors, etc.) may be used in place of the examples provided above.

Handle 802 (e.g., housing 809) may include a recess 854 for receiving a protrusion 954 of handle 902 (e.g., housing 909). Such a configuration may also be reversed. Recess 854 and protrusion 954 may have complementary shapes and may mate via a friction fit or via a retention mechanism 856 (FIG. 9B). For example, recess 854 and protrusion 954 may have a rectangular shape or any suitable other shape. Retention mechanism 856 may include, for example, a tab, a slot, a post, or any other suitable structure.

When first medical device 800 and second medical device 900 are coupled at interface 850, handle 802 may be used to control one or more functions of second medical device 900. Such control may allow for a more seamless experience by a user and may increase ergonomics. In some aspects, handle 902 may include an actuator 815 (e.g., a button or a switch) for switching between control of first medical device 800 and second medical device 900. In aspects, actuators of handle 802 may be used to control steering (articulation), imaging, lighting, fluidics, or other aspects of second medical device 900. For example, one or more connectors 852 and one or more connectors 952 may form electrical or other types (e.g., fluidic) connections. First medical device 800 or second medical device 900 may include one or more of the motor assemblies disclosed above. In some aspects, handle 902 may retain actuators (e.g., knob(s) or joystick(s)) to allow for separate control of second medical device 900. In other aspects, handle 902 may lack such actuators to provide ergonomic benefits and to ease manufacturing.

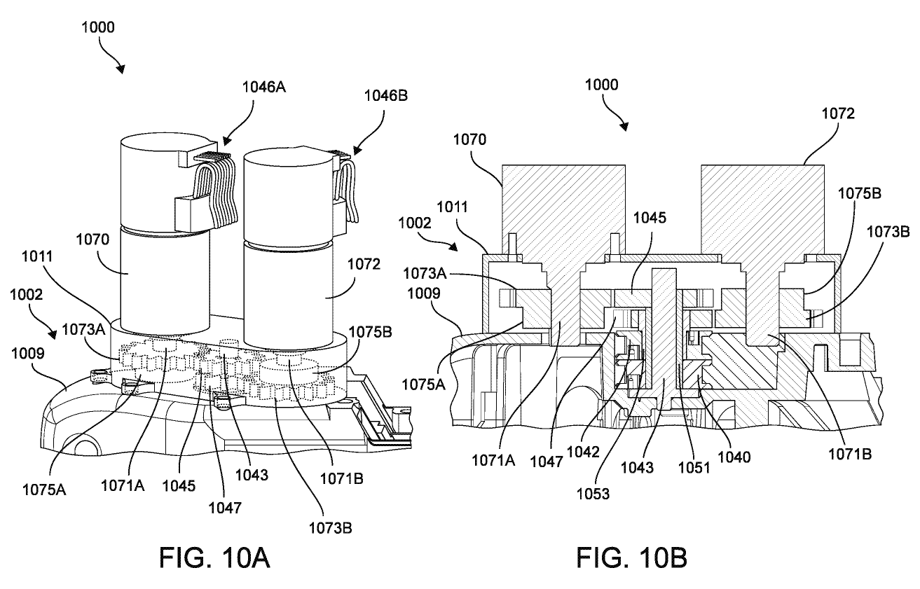

FIGS. 10A and 10B illustrate views of an exemplary medical device 1000. Medical device 1000 may have any or all of the characteristics of medical devices 100, 200, 300, 600, 700, 800, or 900, described above, except as described below. FIG. 10A illustrates a perspective view of medical device 1000 with the internal components shown in broken lines to illustrate their relative position within medical device 1000, and FIG. 10B illustrates a cross-sectional view of medical device 1000 showing the internal arrangement of motor and pulley assemblies, as will be described in further detail below. Medical device 1000 may include an extension having properties of extension 122 or 222, but such an extension is omitted from FIG. 10A in order to show details of medical device 1000. FIGS. 10A and 10B may be referred to concurrently in the following description.

In some aspects, the motor and pulley assemblies of medical device 1000 may operate in any of the operational states described with respect to motor assemblies 321, 421A, 421B, or 521, including fully manual, motor-assisted, fully motorized, or autonomous modes. Medical device 1000 may include a micro-controller having any of the properties of micro-controller 180 described above, including positional memory capabilities, pre-programmed procedural routines, camera stabilization algorithms, force feedback algorithms, and positional holding capabilities.

In some aspects, medical device 1000 may include a display integrated into a handle 1002 or may be configured to communicate with an external display, wherein the display is configured to present any of the display configurations described above, such as display configuration 182 including indicators for articulation position, elevator position, or speed. A shaft extending distally from handle 1002 may include an articulation portion and a distal tip having any of the properties described above with respect to medical devices 100, 200, or 300, including an imaging device, an illumination device, and an elevator.

Handle 1002 may include one or more actuators for controlling the articulation portion, the elevator, or other functions of medical device 1000. The actuators may be any of the actuators previously described with respect to the previous embodiments, including joysticks, knobs, sliders, levers, buttons, switches, or the like. For example, the actuators may be electronically coupled to one or both of PCB boards 1046A, 1046B, which may be electronically coupled to motors 100, 1072, respectively and transmit signals to motors 1070, 1072, respectively in response to actuation. The actuators may be configured to control articulation of an articulation portion of medical device 1000, a position of an elevator at a distal tip, a speed or sensitivity of articulation or actuation, or other functions of medical device 1000.

In some aspects, the actuators may provide proportional control, where a degree of movement of an actuator corresponds to a degree of articulation of the articulation portion or a degree of actuation of another component. Multiple actuators may be used in combination to control different functions of medical device 1000. For example, a first actuator may control articulation of the articulation portion, while a second actuator may control a position of an elevator, and a third actuator may adjust a speed or sensitivity of the articulation or actuation. In other aspects, a first actuator may control one or two articulation directions of the articulation portion, and a second actuator may control one or more articulation directions of the articulation portion.

The components (e.g., internal components) to enable such control include a first gear 1073A and a second gear 1073B positioned near motors 1070 and 1072. For example, a motor shaft 1071A may extend from motor 1070 and be coupled to first gear 1073A. First gear 1073A may have an axis of rotation that is coaxial with motor shaft 1071A. Similarly, a motor shaft 1071B may extend from motor 1072 and be coupled to second gear 1073B. Second gear 1073B may have an axis of rotation that is coaxial with motor shaft 1071B.

First gear 1073A meshes with a third gear 1045. Second gear 1073B meshes with a fourth gear 1047. Thereby, upon rotation of first gear 1073A, third gear 1045 rotates. Similarly, when second gear 1073B rotates, fourth gear 1047 rotates, as well. First gear 1073A and second gear 1073B may be offset from one another along a direction that is approximately parallel to longitudinal axes of motor shafts 1071A and 1071B. For example, one of first gear 1073A and second gear 1073B may be closer to an outside of handle 1002. In other words, one of first gear 1073A and second gear 1073B may have a smaller gap with respect to motor 1070, 1072, respectively. As shown in FIG. 10A, first gear 1073A may be closer to an outside of handle 1002 than second gear 1073B. Stated differently, first gear 1073A may be proximal of second gear 1073B, along a direction that is approximately parallel to longitudinal axes of motor shafts 1071A and 1071B. In other words, first gear 1073A may have a smaller gap relative to motor 1070 than second gear 1073B has relative to motor 1072.

First gear 1073A may be coupled to a first spacer 1075A, and second gear 1073B may be coupled to a second spacer 1075B to maintain proper spacing and alignment. First spacer 1075A may face inward toward a center of handle 1002, and second space 1075B may face outward toward an outside of handle 1002. In other words, first spacer 1075A may be distal to first gear 1073A and relative to second spacer 1075B. along a direction that is approximately parallel to longitudinal axes of motor shafts 1071A and 1071B. Second spacer 1075B may be proximal of second gear 1073B and proximal of first spacer 1075B, along a direction that is approximately parallel to longitudinal axes of motor shafts 1071A and 1071B. In some aspects, gear 1073A and spacer 1075A may be integrally formed, and gear 1073B and spacer 1075B may be integrally formed. The assembly of first gear 1073A and spacer 1075A may be a mirror image to the assembly of second gear 1073B and spacer 1075B along a direction that is approximately parallel to longitudinal axes of motor shafts 1071A and 1071B.

Third gear 1045 and fourth gear 1047 may be configured to move independently from one another. Furthermore, third gear 1045 and fourth gear 1047 may be disposed around a shaft 1043, and share a rotational axis. In other words, third gear 1045 and fourth gear 1047 may be stacked on top of one another. Third gear may be aligned with first gear 1073A along a direction that is approximately parallel to longitudinal axes of motor shafts 1071A and 1071B. Third gear may be aligned with first gear 1073A along a direction that is approximately parallel to longitudinal axes of motor shafts 1071A and 1071B. Fourth gear 1047 may be aligned with first gear 1073B along a direction that is approximately parallel to longitudinal axes of motor shafts 1071A and 1071B. Third gear 1043 may be closer to an outside of handle 1002 than fourth gear 1047. In other words, third gear 1043 may be proximal of fourth gear 1047, along a direction that is approximately parallel to longitudinal axes of motor shafts 1071A and 1071B.

As shown in FIG. 10B, third gear 1045 may be coupled to a first pulley 1040 via a shaft 1051. Third gear 1045 may be fixedly coupled to shaft 1051, and shaft 1051 may be fixedly coupled to first pulley 1040. Fourth gear 1047 may be coupled to second pulley 1042 via a shaft 1053. Fourth gear 1047 may be fixedly coupled to shaft 1053, and shaft 1053 may be fixedly coupled to second pulley 1042. Shaft 1051 may be coaxial to shaft 1043, and Shaft 1053 may be coaxial to connecting shaft 1051 and common shaft 1043. Shaft 1051 may surround shaft 1032, and shaft 1053 may surround shaft 1051.

First pulley 1040 and second pulley 1042 may both rotate about shaft 1043. In other words, each of third gear 1045, fourth gear 1047, first pulley 1040, and second pulley 1042 may be stacked upon one another along a longitudinal axis defined by shaft 1043, which may be approximately parallel to longitudinal axes of motor shafts 1071A and 1071B. Third gear 1045 may be closest to an exterior of handle 1002 (most proximal), and fourth gear 1047 may be next-closet to an exterior of handle 1002 and adjacent to third gear 1045. Second pulley 1042 may be next-closest to an exterior of handle 1002 and adjacent to fourth gear 1047. First pulley 1040 may be the most proximal element of the stack, closest to/furthest into an interior of handle 1002. Shaft 1043 may extend through all of third gear 1045, fourth gear 1047, first pulley 1040, and second pulley 1042.

Accordingly, when motor 1070 is activated, motor shaft 1071A rotates first gear 1073A, which meshes with and rotates third gear 1045, thereby rotating connecting shaft 1051 and first pulley 1040. Similarly, when motor 1072 is activated, motor shaft 1071B rotates second gear 1073B, which meshes with and rotates fourth gear 1047, thereby rotating connecting shaft 1053 and second pulley 1042. Because third gear 1045 and fourth gear 1047 are configured to move independently from one another despite sharing a common rotational axis about shaft 1043, first pulley 1040 and second pulley 1042 may be rotated independently to control different articulation directions of the articulation portion. Pull wires coupled to first pulley 1040 and second pulley 1042 extend distally through the handle 1002 and shaft to the articulation portion, such that rotation of the pulleys causes proximal or distal translation of the pull wires, thereby articulating the articulation portion in corresponding directions.

This configuration allows first pulley 1040 and second pulley 1042 to share a common rotational axis via common shaft 1043 while still being independently rotatable, thereby conserving space within handle 1002 and enabling a more compact handle design.

A number of implementations have been described. Nevertheless, it will be understood that various modifications may be made without departing from the spirit and scope of the disclosure. Accordingly, other implementations are within the scope of the following claims.

1. A medical device, comprising:
a handle;
a shaft extending distally from the handle and including an articulation portion and a distal tip;
an actuator disposed on the handle and configured to control articulation of the articulation portion;
a pulley disposed within the handle;
a first wire and a second wire, wherein a proximal end of each of the first wire and the second wire is coupled to the pulley, and wherein a distal end of each of the first wire and the second wire extends to the articulation portion; and
a motor disposed within the handle and operatively connected to the pulley, wherein, in response to manipulation of the actuator in a first actuation direction, the motor is configured to rotate the pulley, causing the proximal end of the first wire to move proximally and a proximal end of the second wire to move distally, such that the articulation portion articulates in a first articulation direction.

2. The medical device of claim 1, wherein the pulley is a first pulley and the motor is a first motor, and wherein the medical device further comprises:
a second pulley disposed within the handle;
a second motor disposed within the handle and operatively connected to the second pulley; and
a third wire and a fourth wire, wherein a proximal end of each of the third wire and the fourth wire is coupled to the second pulley, and wherein a distal end of each of the third wire and the fourth wire extends to the articulation portion,
wherein, in response to manipulation of the actuator in a second actuation direction, that is different from the first actuation direction, the second motor rotates the second pulley, causing the third wire to translate proximally and the fourth wire to translate distally, thereby articulating the articulation portion in a second articulation direction, that is different from the first articulation direction.

3. The medical device of claim 2, wherein, in response to manipulation of the actuator in a third actuation direction, each of the first pulley and the second pulley are rotated, thereby articulating the articulation portion in a third actuation direction, that is different from the first actuation direction or the second actuation direction.

4. The medical device of claim 2, wherein the first pulley and the second pulley are adjacent to one another and have rotation axes that are parallel to one another but not coaxial.

5. The medical device of claim 1, wherein the pulley includes an axle extending from a first side of the handle to a second side of the handle, wherein the motor is configured to rotate the pulley by rotating the axle.

6. The medical device of claim 1, wherein the actuator is a joystick.

7. The medical device of claim 1, wherein a degree of movement of the actuator corresponds to a degree of articulation of the articulation portion.

8. The medical device of claim 1, wherein the actuator is a first actuator, and the motor is a pulley motor, and wherein the medical device further comprises:
an elevator disposed at the distal tip;
a second actuator disposed on the handle, wherein the second actuator is configured to control a position of the elevator; and
an elevator motor configured to actuate the elevator in response to actuation of the second actuator.

9. The medical device of claim 8, wherein the medical device further comprises:
an arm disposed within the handle; and
an elevator pull wire having a distal end connected to the elevator and a proximal end directly or indirectly connected to the arm,
wherein the elevator motor is configured to rotate the arm to move the elevator pull wire proximally or distally to actuate the elevator.

10. The medical device of claim 2, wherein the first pulley and the second pulley rotate about a same rotational axis.

11. The medical device of claim 8, further comprising a third actuator, wherein the third actuator is configured to adjust a speed of articulation of the articulation portion, a speed of actuation of the elevator, or both.

12. The medical device of claim 11, wherein the third actuator is a knob, and wherein an axis of rotation of the knob is approximately parallel to a longitudinal axis of the handle.

13. The medical device of claim 11, further comprising a display integrated into the handle and configured to provide visual feedback regarding one or more of a position of the articulation portion, a position of the elevator, or a speed.

14. The medical device of claim 13, wherein the visual feedback regarding the position of the articulation portion includes a quadrant-based representation.

15. The medical device of claim 14, wherein the visual feedback includes a first symbol regarding the position of the articulation portion and a second symbol regarding a previous position of the articulation portion.

16. A medical method comprising:
receiving information related to a position of an articulation portion of a medical device; and
displaying the position on of the articulation portion on a graphical user interface, wherein the graphical user interface includes a quadrant illustration, a first symbol representing a current position of the articulation portion, and a second symbol representing a previous position of the articulation portion.

17. The medical method of claim 16, wherein the graphical user interface is displayed on a screen integrated into a handle of the medical device.

18. The medical method of claim 16, further comprising automatically moving the articulation portion to the previous position.

19. A medical device, comprising:
a handle;
a shaft extending distally from the handle and including an articulation portion and a distal tip, wherein the distal tip includes an elevator;
a first actuator disposed on the handle and configured to control articulation of the articulation portion;
a second actuator disposed on the handle and configured to control a position of the elevator; and
a motor configured to actuate the elevator in response to actuation of the second actuator.

20. The medical device of claim 19, further comprising a display integrated into the handle and configured to provide visual feedback regarding the position of the elevator.
